(19) United States
(12) Patent Application Publication
(10) Pub. No.: US 2026/0120833 A1
(43) Pub. Date: Apr. 30, 2026

(54) METHODS AND SYSTEMS THAT CONTINUOUSLY PROCESS DATA TO PERIODICALLY DEVELOP AND REFINE COMPUTATIONAL MODELS THAT ARE USED TO AUTOMATICALLY GENERATE WHOLE-BODY, SPECIFIC-ORGAN-SYSTEM, MULTI-ORGAN-SYSTEM-CONDITION, AND FUNCTIONALITY HEALTH-ENTROPY-METRIC VALUES

(71) Applicant: Shabnam Salimi, Shoreline, WA (US)

(72) Inventor: Shabnam Salimi, Shoreline, WA (US)

(21) Appl. No.: 19/371,986

(22) Filed: Oct. 28, 2025

Related U.S. Application Data
(60) Provisional application No. 63/712,912, filed on Oct. 28, 2024.

(51) Int. Cl. G16H 15/00

(52) U.S. Cl. CPC G16H 15/00

(57) ABSTRACT

The current document is directed to an automated system that continuously collects and processes physiological, functional, and health data, processes the collected data to continuously develop and refine computational models that generate, from the collected data, multiple health-entropy metrics for the individual's whole-body, for specific-organ-systems, for specific multi-organ-system conditions, and for specific functionalities. The health-entropy metrics are transmitted to organizations, professionals, and individuals for monitoring individuals' health, for diagnosing diseases and conditions, for developing therapies and treatments for individuals as well as generally applicable therapies and treatments, for supporting basic research in pharmaceutical development, biomarker selection, diagnosing and monitoring various diseases and conditions, and public health monitoring, and for use by individuals for diet planning, accessing healthcare services, making lifestyle selections and adjustments, and monitoring therapies and treatments.

This application claims the benefit of Provisional Patent Application 63/712,912 filed Oct. 28, 2024, the contents of which is hereby expressly incorporated by reference in its entirety.

### TECHNICAL FIELD

The current document is directed to health monitoring, medical diagnosis, medical-treatment development and monitoring, and medical, pharmaceutical, and public-health research and, in particular, to an automated system that continuously collects and processes health-related data, periodically develops and refines models that generate, from the processed health-related data, whole-body, specific-organ-system, multi-organ-system condition, and functionality health-entropy indications that are used, by downstream systems and professionals, to monitor an individual's health, to devise therapies and treatments, to carry out basic research, drug development, and biomarker selection for various diseases and conditions, and that are used, by individuals, for diet planning, healthcare access, lifestyle selections and adjustments, and medical-treatment monitoring.

### BACKGROUND

Current medical practice focuses on diagnosis and treatment of discrete diseases and disorders. Medical practice and medical research often fail to consider the implications of multiple, concurrent diseases and health conditions within an individual. These implications may include increased or decreased susceptibilities to additional diseases and disorders, increases or decreases in the rates of disease progression, non-linear physiological responses, increases or decreases in resilience and immune function, increased or decreased rates of decline related to aging, and many additional complexities associated with humans.

The human organism is hierarchically organized, is enormously complex at many different levels, from the molecular level up through biomolecular complexes, cellular components, cells, components of organs, entire organs, and organ systems, and functionalities. It can be difficult or impossible to qualitatively assess the physiological states of individual components of the human organism at different levels of this hierarchy, and more difficult still to attempt to quantify these physiological states and compare them both within an individual and across multiple individuals and organs. It is also difficult to understand, and to develop metrics to assess and monitor, progressive phenomena such as aging, declining functionalities, disease progression, and the combined effects of multiple different diseases and conditions over time. A further complexity is that each individual is unique, from the individual's molecular components all the way up to mental and physical processes and functionalities. The responses of one individual to physical challenges, diseases, disorders, and treatments may differ greatly from those of another individual to the same physical challenges, diseases, disorders, and therapies and treatments.

As further explained, below, the concept of health entropy provides a qualitative indication of the general physiological state of an individual that spans organ systems as well as the whole body. A number of metrics related to overall health entropy have been proposed and used. However, these metrics are generally based on the occurrences of discrete clinical diseases, fail to take account of sub-clinical disorders and conditions and multiple concurrent diseases and health conditions, and generally apply to an organism as a whole rather than to individual organs and organ systems. Moreover, the effects of health entropy on functionality and physical and cognitive ability have been overlooked. As a result, the current metrics are generally of limited value to medical professionals, researchers, and individuals. Medical professionals, researchers, and individuals have recognized the deficiencies of currently available health-entropy metrics and of single-disease-oriented medical-diagnosis-and-treatment practices.

### SUMMARY

The current document is directed to an automated system that continuously collects and processes physiological, functional, and health data, processes the collected data to continuously develop and refine computational models that generate, from the collected data, multiple health-entropy metrics for the individual's whole-body, for specific-organ-systems, for specific multi-organ-system conditions, and for specific functionalities. The health-entropy metrics are transmitted to organizations, professionals, and individuals for monitoring individuals' health, for diagnosing diseases and conditions, for developing therapies and treatments for individuals as well as generally applicable therapies and treatments, for supporting basic research in pharmaceutical development, disease prevention, biomarker selection, diagnosing and monitoring various diseases and conditions, and public health monitoring, and for use by individuals for diet planning, accessing healthcare services, making lifestyle selections and adjustments, and monitoring therapies, treatments, and disease-prevention strategies.

### BRIEF DESCRIPTION OF THE DRAWINGS

FIG. 1 provides a general architectural diagram for various types of computers.

FIG. 2 illustrates an Internet-connected distributed computing system.

FIG. 3 illustrates cloud computing.

FIG. 4 illustrates generalized hardware and software components of a general-purpose computer system, such as a general-purpose computer system having an architecture similar to that shown in FIG. 1.

FIGS. 5A-B illustrate two types of virtual machine and virtual-machine execution environments.

FIGS. 6-7 illustrate several meanings of the term “entropy.”

FIG. 8 illustrates the relationship of chronological age to health entropy and the relationship of biological age to both health entropy and chronological age.

FIG. 9 illustrates one objective of the currently disclosed methods and systems.

FIG. 10 provides a high-level illustration of the currently disclosed system.

FIGS. 11-25 provide an overview of probability and statistics that may be useful to a reader for understanding the computations carried out by the currently disclosed methods and systems.

FIG. 26 provides a control-flow diagram for the server application.

FIG. 27 provides a control-flow diagram for the client-side application that runs on user computers in certain implementations of the currently disclosed system.

FIG. 28A illustrates the type of raw data and processed data that is used by the current system for generating computational models and using the computational models to generate health-entropy-metric values and other derived values.

FIG. 28B provides a control-flow diagram for the new-data handler called in step 2616 of FIG. 26.

FIG. 29 illustrates the contents of the overall computational model used to generate health-entropy-metric values and other intermediate values from health data and other information furnished by users in one implementation of the currently disclosed system.

FIG. 30 provides a control-flow diagram for the routine compute_model called in step 7822 of FIG. 28.

FIGS. 31A-D provide a control-flow diagram for the routine adjust_model called in step 3005 of FIG. 30.

FIG. 32 provides a control-flow diagram for the routine computed_values_request called in step 2620 of FIG. 26.

FIG. 33 illustrates the health-entropy-metric values and other computed values that are calculated by the server application.

FIGS. 34A-B provide a control-flow diagram for the routine compute_values called in step 3216 of FIG. 32.

FIGS. 35A-F provide details for each of the Bayesian-inference-based regressions used to generate the computational models for the various health-entropy-metric values and other computed values by the currently disclosed methods and systems.

FIG. 36 shows the disability events used in one implementation of the currently disclosed methods and systems to generate the disability index, disability clock, and disability biological age.

FIGS. 37A-I show the organ systems and multi-organ-system conditions, and associated health conditions and diseases, used in one implementation of the currently disclosed methods and systems.

### DETAILED DESCRIPTION

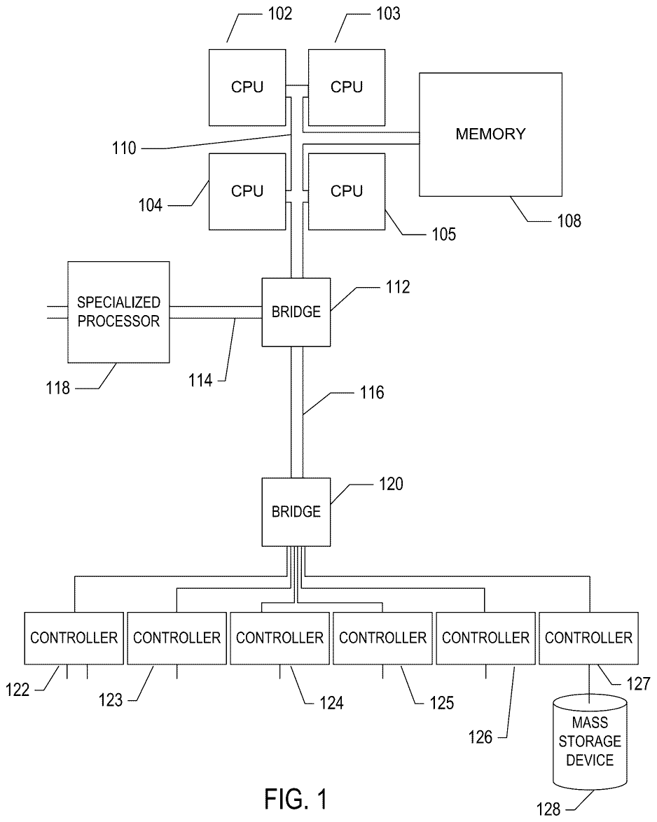

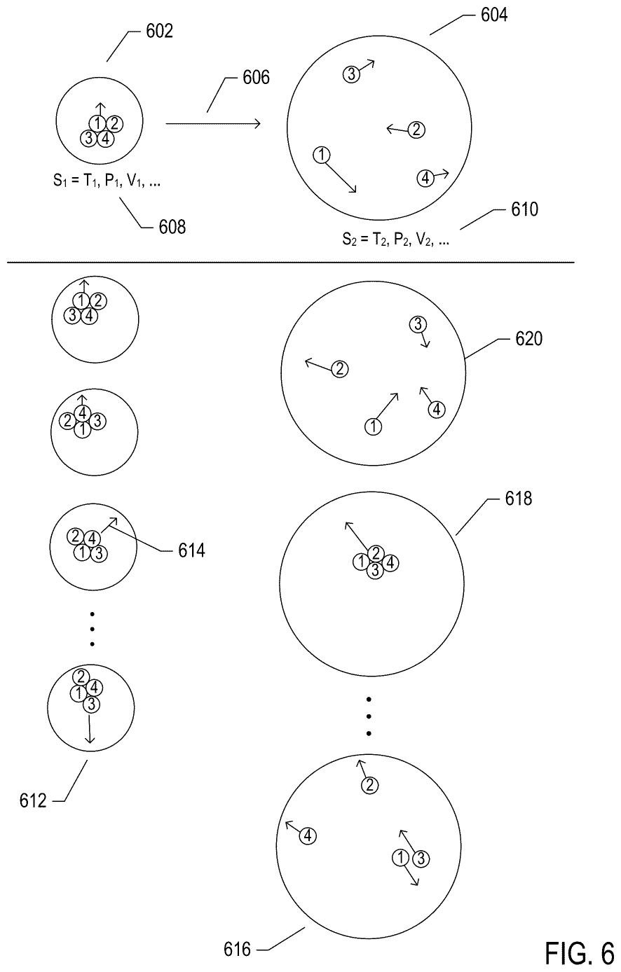

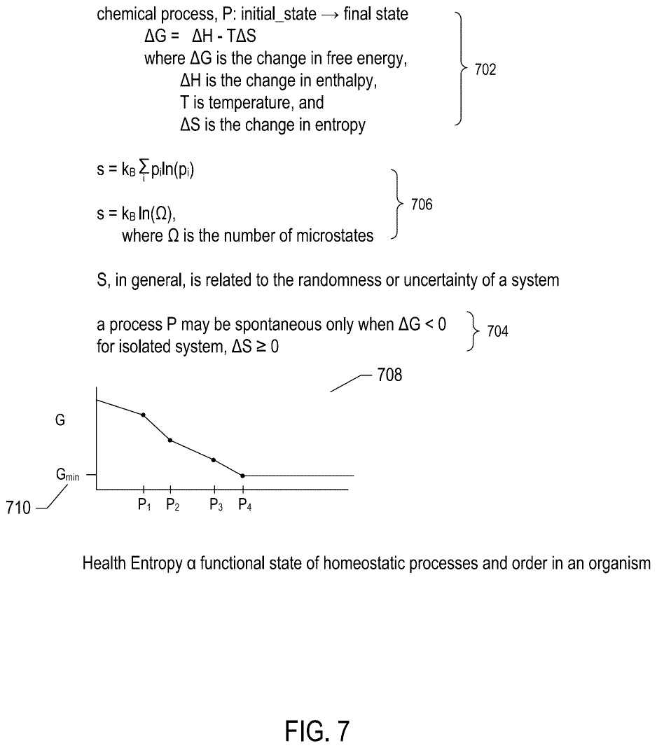

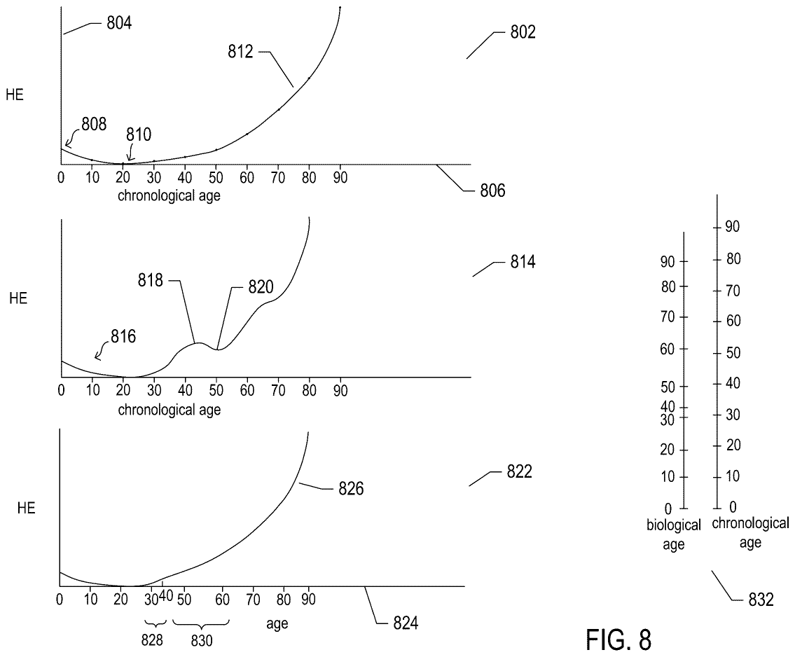

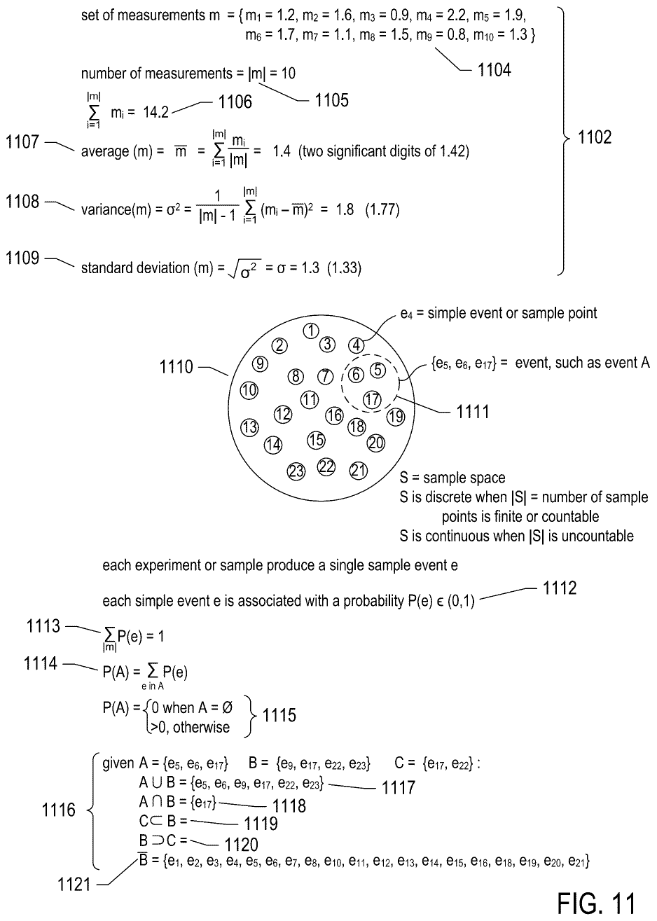

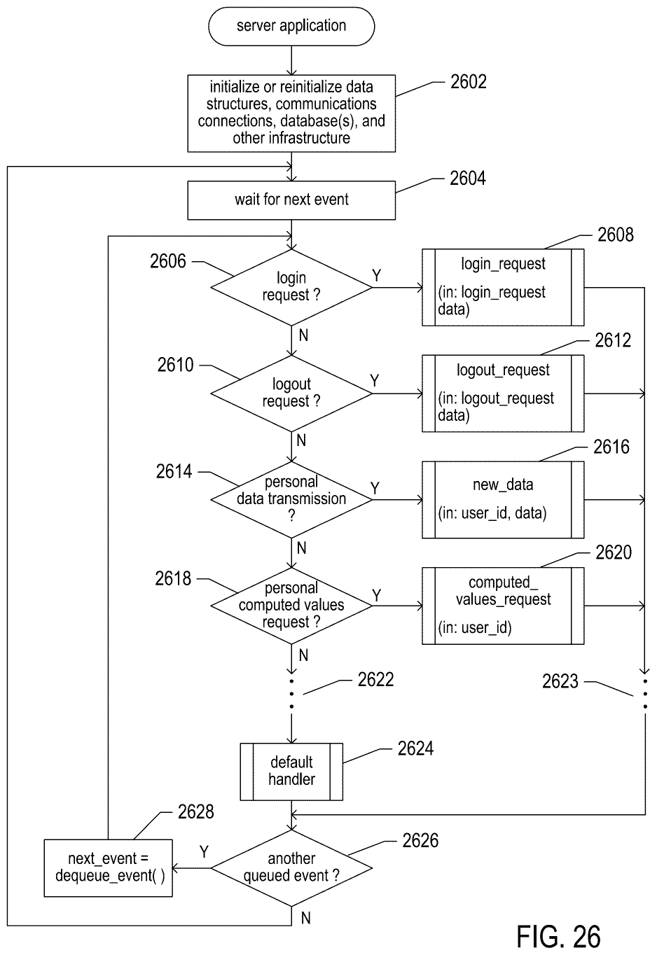

The current application is directed to an automated system that continuously collects and processes physiological, functional, and health data, processes the collected data to continuously develop and refine computational models that generate, from an individual's physiological, functional, and health data, multiple health-entropy metrics for the individual's whole-body, for specific-organ-systems, and for specific functionalities and conditions. In a first subsection, below, a detailed description of computer hardware, complex computational systems, and virtualization is provided with reference to FIGS. 1-5B. In a second subsection, an overview of health entropy and biological age is provided with reference to FIGS. 6-7. A third subsection provides an overview of currently disclosed methods and systems with reference to FIGS. 8-10. A fourth subsection provides an overview of probability and statistics with reference to FIGS. 11-25. Finally, in a fifth subsection, the currently disclosed methods and systems are discussed with reference to FIGS. 26-37I.

### Computer Hardware, Complex Computational Systems, and Virtualization

FIG. 1 provides a general architectural diagram for various types of computers. The computer system contains one or multiple central processing units (“CPUs”) 102-105, one or more electronic memories 108 interconnected with the CPUs by a CPU/memory-subsystem bus 110 or multiple busses, a first bridge 112 that interconnects the CPU/memory-subsystem bus 110 with additional busses 114 and 116, or other types of high-speed interconnection media, including multiple, high-speed serial interconnects. These busses or serial interconnections, in turn, connect the CPUs and memory with specialized processors, such as a graphics processor 118, and with one or more additional bridges 120, which are interconnected with high-speed serial links or with multiple controllers 122-127, such as controller 127, that provide access to various different types of mass-storage devices 128, electronic displays, input devices, and other such components, subcomponents, and computational resources. It should be noted that computer-readable data-storage devices include optical and electromagnetic disks, electronic memories, and other physical data-storage devices. Those familiar with modern science and technology appreciate that electromagnetic radiation and propagating signals do not store data for subsequent retrieval and can transiently “store” only a byte or less of information per mile, far less information than needed to encode even the simplest of routines.

Of course, there are many different types of computer-system architectures that differ from one another in the number of different memories, including different types of hierarchical cache memories, the number of processors and the connectivity of the processors with other system components, the number of internal communications busses and serial links, and in many other ways. However, computer systems generally execute stored programs by fetching instructions from memory and executing the instructions in one or more processors. Computer systems include general-purpose computer systems, such as personal computers (“PCs”), various types of servers and workstations, and higher-end mainframe computers, but may also include a plethora of various types of special-purpose computing devices, including data-storage systems, communications routers, network nodes, tablet computers, and mobile telephones.

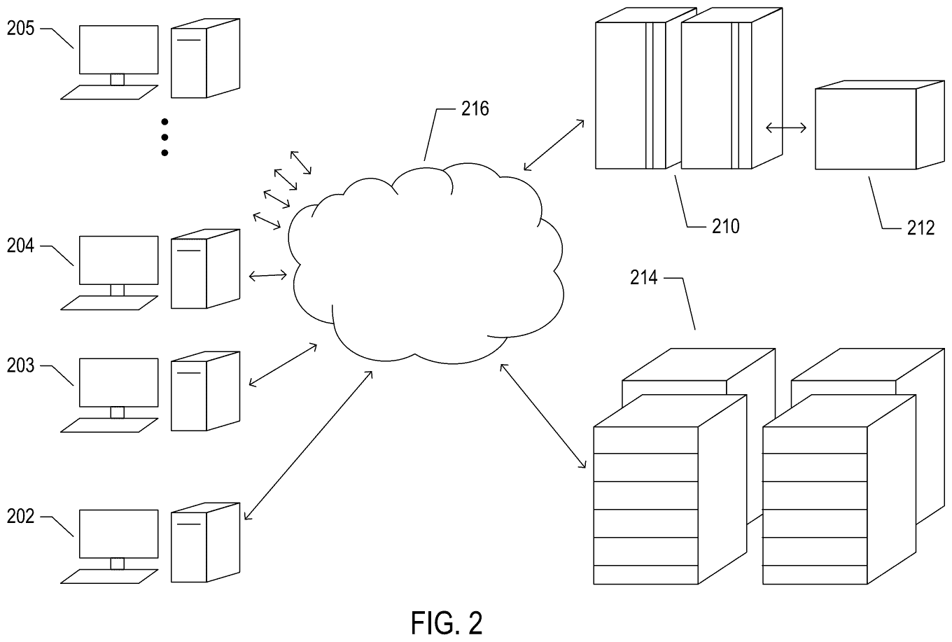

FIG. 2 illustrates an Internet-connected distributed computing system. As communications and networking technologies have evolved in capability and accessibility, and as the computational bandwidths, data-storage capacities, and other capabilities and capacities of various types of computer systems have steadily and rapidly increased, much of modern computing now generally involves large distributed systems and computers interconnected by local networks, wide-area networks, wireless communications, and the Internet. FIG. 2 shows a typical distributed system in which a large number of PCs 202-205, a high-end distributed mainframe system 210 with a large data-storage system 212, and a large computer center 214 with large numbers of rack-mounted servers or blade servers all interconnected through various communications and networking systems that together comprise the Internet 216. Such distributed computing systems provide diverse arrays of functionalities. For example, a PC user sitting in a home office may access hundreds of millions of different web sites provided by hundreds of thousands of different web servers throughout the world and may access high-computational-bandwidth computing services from remote computer facilities for running complex computational tasks.

Until recently, computational services were generally provided by computer systems and data centers purchased, configured, managed, and maintained by service-provider organizations. For example, an e-commerce retailer generally purchased, configured, managed, and maintained a data center including numerous web servers, back-end computer systems, and data-storage systems for serving web pages to remote customers, receiving orders through the web-page interface, processing the orders, tracking completed orders, and other myriad different tasks associated with an e-commerce enterprise.

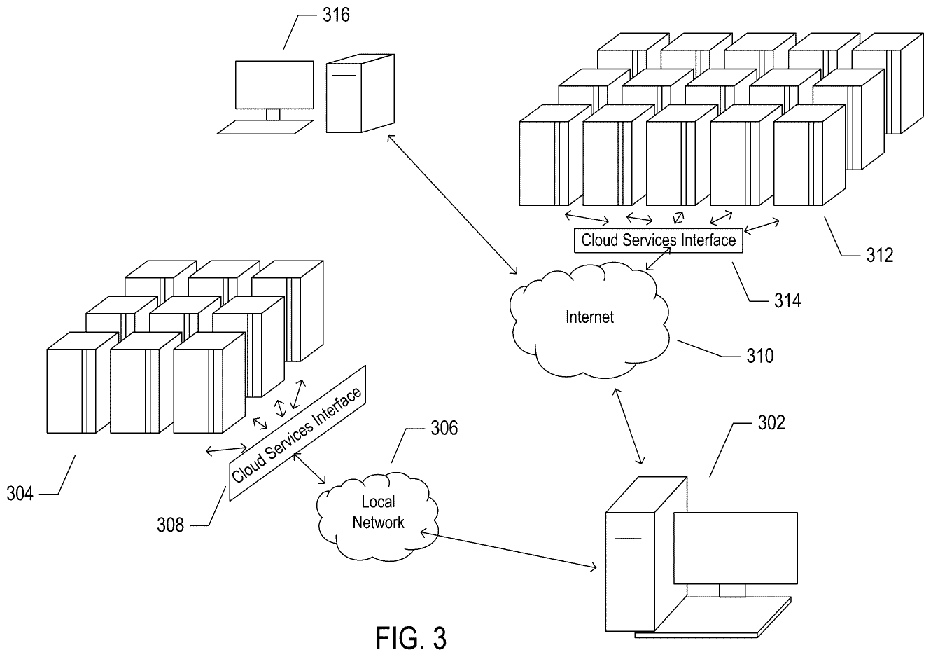

FIG. 3 illustrates cloud computing. In the recently developed cloud-computing paradigm, computing cycles and data-storage facilities are provided to organizations and individuals by cloud-computing providers. In addition, larger organizations may elect to establish private cloud-computing facilities in addition to, or instead of, subscribing to computing services provided by public cloud-computing service providers. In FIG. 3, a system administrator for an organization, using a PC 302, accesses the organization's private cloud 304 through a local network 306 and private-cloud interface 308 and also accesses, through the Internet 310, a public cloud 312 through a public-cloud services interface 314. The administrator can, in either the case of the private cloud 304 or public cloud 312, configure virtual computer systems and even entire virtual data centers and launch execution of application programs on the virtual computer systems and virtual data centers in order to carry out any of many different types of computational tasks. As one example, a small organization may configure and run a virtual data center within a public cloud that executes web servers to provide an e-commerce interface through the public cloud to remote customers of the organization, such as a user viewing the organization's e-commerce web pages on a remote user system 316.

Cloud-computing facilities are intended to provide computational bandwidth and data-storage services much as utility companies provide electrical power and water to consumers. Cloud computing provides enormous advantages to small organizations without the resources to purchase, manage, and maintain in-house data centers. Such organizations can dynamically add and delete virtual computer systems from their virtual data centers within public clouds in order to track computational-bandwidth and data-storage needs, rather than purchasing sufficient computer systems within a physical data center to handle peak computational-bandwidth and data-storage demands. Moreover, small organizations can completely avoid the overhead of maintaining and managing physical computer systems, including hiring and periodically retraining information-technology specialists and continuously paying for operating-system and database-management-system upgrades. Furthermore, cloud-computing interfaces allow for easy and straightforward configuration of virtual computing facilities, flexibility in the types of applications and operating systems that can be configured, and other functionalities that are useful even for owners and administrators of private cloud-computing facilities used by a single organization.

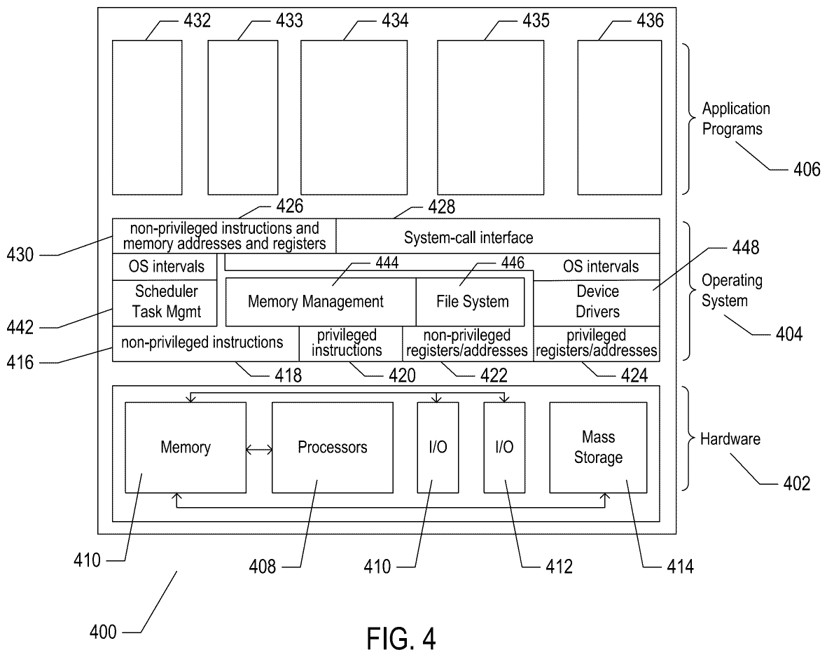

FIG. 4 illustrates generalized hardware and software components of a general-purpose computer system, such as a general-purpose computer system having an architecture similar to that shown in FIG. 1. The computer system 400 is often considered to include three fundamental layers: (1) a hardware layer or level 402; (2) an operating-system layer or level 404; and (3) an application-program layer or level 406. The hardware layer 402 includes one or more processors 408, system memory 410, various different types of input-output (“I/O”) devices 410 and 412, and mass-storage devices 414. Of course, the hardware level also includes many other components, including power supplies, internal communications links and busses, specialized integrated circuits, many different types of processor-controlled or microprocessor-controlled peripheral devices and controllers, and many other components. The operating system 404 interfaces to the hardware level 402 through a low-level operating system and hardware interface 416 generally comprising a set of non-privileged computer instructions 418, a set of privileged computer instructions 420, a set of non-privileged registers and memory addresses 422, and a set of privileged registers and memory addresses 424. In general, the operating system exposes non-privileged instructions, non-privileged registers, and non-privileged memory addresses 426 and a system-call interface 428 as an operating-system interface 430 to application programs 432-436 that execute within an execution environment provided to the application programs by the operating system. The operating system, alone, accesses the privileged instructions, privileged registers, and privileged memory addresses. By reserving access to privileged instructions, privileged registers, and privileged memory addresses, the operating system can ensure that application programs and other higher-level computational entities cannot interfere with one another's execution and cannot change the overall state of the computer system in ways that could deleteriously impact system operation. The operating system includes many internal components and modules, including a scheduler 442, memory management 444, a file system 446, device drivers 448, and many other components and modules. To a certain degree, modern operating systems provide numerous levels of abstraction above the hardware level, including virtual memory, which provides to each application program and other computational entities a separate, large, linear memory-address space that is mapped by the operating system to various electronic memories and mass-storage devices. The scheduler orchestrates interleaved execution of various different application programs and higher-level computational entities, providing to each application program a virtual, stand-alone system devoted entirely to the application program. From the application program's standpoint, the application program executes continuously without concern for the need to share processor resources and other system resources with other application programs and higher-level computational entities. The device drivers abstract details of hardware-component operation, allowing application programs to employ the system-call interface for transmitting and receiving data to and from communications networks, mass-storage devices, and other I/O devices and subsystems. The file system 436 facilitates abstraction of mass-storage-device and memory resources as a high-level, easy-to-access, file-system interface. Thus, the development and evolution of the operating system has resulted in the generation of a type of multi-faceted virtual execution environment for application programs and other higher-level computational entities.

While the execution environments provided by operating systems have proved to be an enormously successful level of abstraction within computer systems, the operating-system-provided level of abstraction is nonetheless associated with difficulties and challenges for developers and users of application programs and other higher-level computational entities. One difficulty arises from the fact that there are many different operating systems that run within various different types of computer hardware. In many cases, popular application programs and computational systems are developed to run on only a subset of the available operating systems and can therefore be executed within only a subset of the various different types of computer systems on which the operating systems are designed to run. Often, even when an application program or other computational system is ported to additional operating systems, the application program or other computational system can nonetheless run more efficiently on the operating systems for which the application program or other computational system was originally targeted. Another difficulty arises from the increasingly distributed nature of computer systems. Although distributed operating systems are the subject of considerable research and development efforts, many of the popular operating systems are designed primarily for execution on a single computer system. In many cases, it is difficult to move application programs, in real time, between the different computer systems of a distributed computing system for high-availability, fault-tolerance, and load-balancing purposes. The problems are even greater in heterogeneous distributed computing systems which include different types of hardware and devices running different types of operating systems. Operating systems continue to evolve, as a result of which certain older application programs and other computational entities may be incompatible with more recent versions of operating systems for which they are targeted, creating compatibility issues that are particularly difficult to manage in large distributed systems.

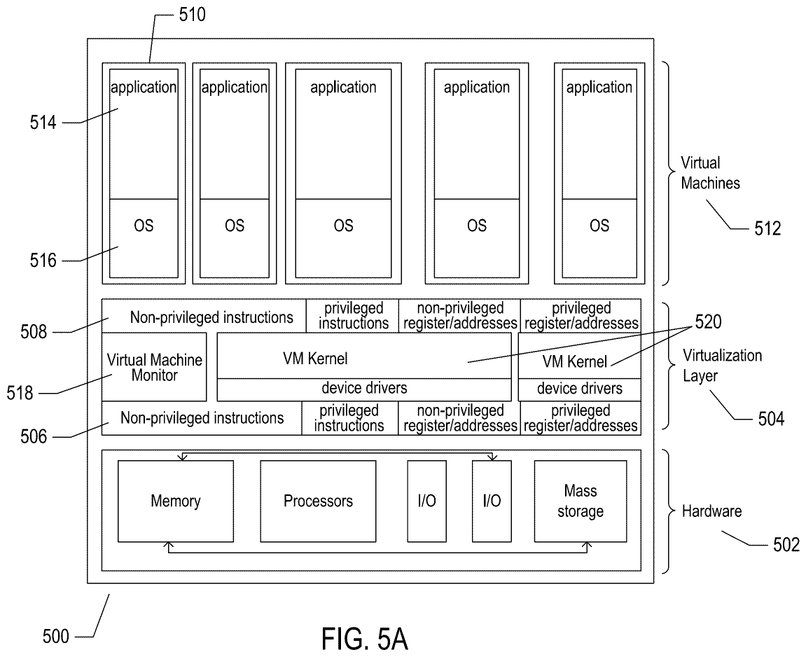

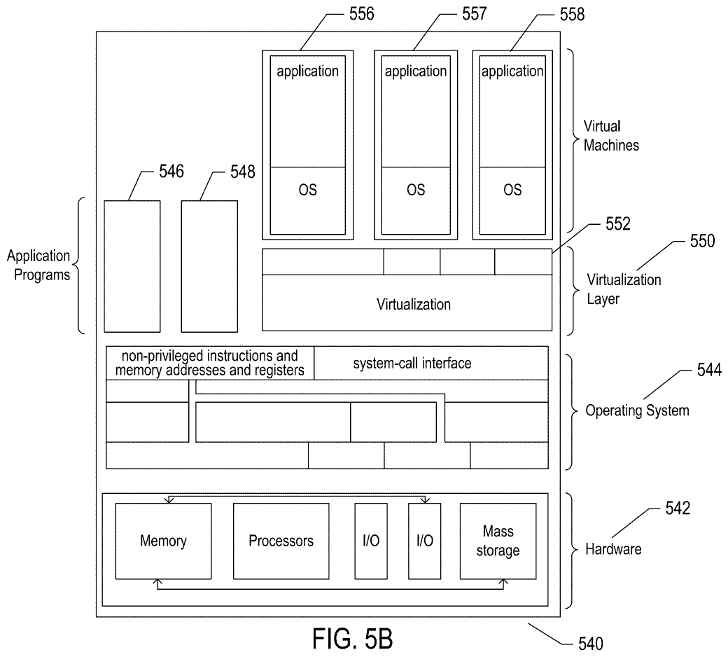

For all of these reasons, a higher level of abstraction, referred to as the “virtual machine,” has been developed and evolved to further abstract computer hardware in order to address many difficulties and challenges associated with traditional computing systems, including the compatibility issues discussed above. FIGS. 5A-D illustrate several types of virtual machine and virtual-machine execution environments. FIGS. 5A-B use the same illustration conventions as used in FIG. 4. FIG. 5A shows a first type of virtualization. The computer system 500 in FIG. 5A includes the same hardware layer 502 as the hardware layer 402 shown in FIG. 4. However, rather than providing an operating system layer directly above the hardware layer, as in FIG. 4, the virtualized computing environment illustrated in FIG. 5A features a virtualization layer 504 that interfaces through a virtualization-layer/hardware-layer interface 506, equivalent to interface 416 in FIG. 4, to the hardware. The virtualization layer provides a hardware-like interface 508 to a number of virtual machines, such as virtual machine 510, executing above the virtualization layer in a virtual-machine layer 512. Each virtual machine includes one or more application programs or other higher-level computational entities packaged together with an operating system, referred to as a “guest operating system,” such as application 514 and guest operating system 516 packaged together within virtual machine 510. Each virtual machine is thus equivalent to the operating-system layer 404 and application-program layer 406 in the general-purpose computer system shown in FIG. 4. Each guest operating system within a virtual machine interfaces to the virtualization-layer interface 508 rather than to the actual hardware interface 506. The virtualization layer partitions hardware resources into abstract virtual-hardware layers to which each guest operating system within a virtual machine interfaces. The guest operating systems within the virtual machines, in general, are unaware of the virtualization layer and operate as if they were directly accessing a true hardware interface. The virtualization layer ensures that each of the virtual machines currently executing within the virtual environment receive a fair allocation of underlying hardware resources and that all virtual machines receive sufficient resources to progress in execution. The virtualization-layer interface 508 may differ for different guest operating systems. For example, the virtualization layer is generally able to provide virtual hardware interfaces for a variety of different types of computer hardware. This allows, as one example, a virtual machine that includes a guest operating system designed for a particular computer architecture to run on hardware of a different architecture. The number of virtual machines need not be equal to the number of physical processors or even a multiple of the number of processors.

The virtualization layer includes a virtual-machine-monitor module 518 (“VMM”) that virtualizes physical processors in the hardware layer to create virtual processors on which each of the virtual machines executes. For execution efficiency, the virtualization layer attempts to allow virtual machines to directly execute non-privileged instructions and to directly access non-privileged registers and memory. However, when the guest operating system within a virtual machine accesses virtual privileged instructions, virtual privileged registers, and virtual privileged memory through the virtualization-layer interface 508, the accesses result in execution of virtualization-layer code to simulate or emulate the privileged resources. The virtualization layer additionally includes a kernel module 520 that manages memory, communications, and data-storage machine resources on behalf of executing virtual machines (“VM kernel”). The VM kernel, for example, maintains shadow page tables on each virtual machine so that hardware-level virtual-memory facilities can be used to process memory accesses. The VM kernel additionally includes routines that implement virtual communications and data-storage devices as well as device drivers that directly control the operation of underlying hardware communications and data-storage devices. Similarly, the VM kernel virtualizes various other types of I/O devices, including keyboards, optical-disk drives, and other such devices. The virtualization layer essentially schedules execution of virtual machines much like an operating system schedules execution of application programs, so that the virtual machines each execute within a complete and fully functional virtual hardware layer.

FIG. 5B illustrates a second type of virtualization. In FIG. 5B, the computer system 540 includes the same hardware layer 542 and software layer 544 as the hardware layer 402 shown in FIG. 4. Several application programs 546 and 548 are shown running in the execution environment provided by the operating system. In addition, a virtualization layer 550 is also provided, in computer 540, but, unlike the virtualization layer 504 discussed with reference to FIG. 5A, virtualization layer 550 is layered above the operating system 544, referred to as the “host OS,” and uses the operating system interface to access operating-system-provided functionality as well as the hardware. The virtualization layer 550 comprises primarily a VMM and a hardware-like interface 552, similar to hardware-like interface 508 in FIG. 5A. The virtualization-layer/hardware-layer interface 552, equivalent to interface 416 in FIG. 4, provides an execution environment for a number of virtual machines 556-558, each including one or more application programs or other higher-level computational entities packaged together with a guest operating system.

### An Overview of Health Entropy and Biological Age

The concept of entropy is fundamental to classical thermodynamics, statistical mechanics, information science, and many other fields. In the field of health, entropy is related to a random expansion of health deterioration. FIGS. 6-7 illustrate several meanings of the term “entropy.” FIG. 6 shows a hypothetical scenario to illustrate one meaning of the term “entropy.” This scenario considers an isolated system 602 and 604, with the system in an initial state 602 and transitioning to a second state 604 by a state-change process 606. In this simplistic, hypothetical scenario, the system can be considered to be a flexible spherical membrane containing four particles that is, in turn, contained within a larger rigid container isolated from its external environment. In the first state 602, the particles are bound together in a rigid structure, similar to molecules in a solid-state substance. In the second state 604, the particles are no longer bound together and are therefore free to move independently, similar to a gaseous state of the substance. Each state can be considered to be specified, or described, by a collection of state variables, as indicated by expressions 608 and 610, with the state variables including temperature, pressure, volume, and other observable quantities. Since the hypothetical system is isolated, the energy and mass of the system is conserved.

In classical thermodynamics, entropy is a state variable and the change in entropy can be computed as a small amount transferred to a system, during a reversible process, divided by temperature. In statistical mechanics, the entropy associated with a state of the system is related to the number of different micro-states available to the system at a particular system energy. In the current example, each micro state is defined by the positions and momenta of the four particles. For the first state, as illustrated in column 612, the four particles are rigidly bound together, with the different micro-states including states where the bound four particles have different positions, rotational orientations, and momenta, where the momenta are represented by arrows, such as arrow 614. In the second state, the micro-states are similarly represented in column 616. However, in the second state, there are many more possible micro-states available to the system, since the four particles are no longer bound together. They may, at a given point in time, occupy positions in which they appear to be bound together, such as in micro state 618, but, in other micro-states, such as micro state 620, may occupy very different positions and may each have different momenta. Thus, the entropy of the system in the second state 604 is much greater than the entropy of the system in the first state 602. In both classical thermodynamics and statistical mechanics, as well as in many other fields, including health, entropy is considered to represent an indication of the level of randomness, uncertainty, and/or disorder in whatever system or environment is being considered, with greater levels of randomness, disorder, or uncertainty associated with higher entropy values.

As shown in the initial expressions 702 at the top of FIG. 7, for state transitions associated with chemical processes, the free-energy change ΔG for the process is computed as the change in enthalpy, ΔH, minus the temperature times the change in entropy, TΔS. Chemical processes spontaneously occur only when ΔG has a negative value. The change in entropy for spontaneous processes in an isolated system is greater than or equal to 0 (704). Thus, a chemical process may be spontaneous when both the enthalpy change is negative or, in other words, when the process generates heat that is transferred to its environment, and when the entropy change is positive or, in other words, the process results in an increase in randomness or uncertainty, or when the sum of the enthalpy change and the temperature times the entropy change is negative, despite the enthalpy change being positive or the entropy change being negative. This is consistent with the statistical mechanics definition of entropy as being proportional to the number of micro-states available to a system, as indicated in expressions 706, where kB is the Boltzmann constant, i is a micro state, and pᵢ is the probability of micro state i being occupied. One way to look at the free energy is that, as shown in plot 708, for a sequence of spontaneous processes P₁, P₂, P₃, P₄, the free energy of the system decreases towards a minimum value 710 at which the entropy has a maximum value for the final temperature of the system, which is referred to as the equilibrium state of the system.

The phrase “health entropy” is related to the concept of entropy in classical thermodynamics, statistical mechanics, information sciences, and other fields. The human organism is an extremely complex, hierarchical structure, as mentioned above. Moreover, the human organism is dynamic and an open system that continuously exchanges mass and energy with its environment. The human organism, as with all organisms, is therefore an open system that occupies a decidedly non-equilibrium state while alive, continuously extracting energy from its environment in order to maintain a comparatively low-entropy state represented by the precisely ordered monomers in biopolymers, including DNA, RNA, proteins, carbohydrate polymers, lipids, and other types of biopolymers, by the structures of macromolecular complexes, cellular organelles, cells, components of organs, and organ systems, and by the extremely complex networks of chemical, electrochemical, and mechanical processes that function homeostatically to maintain the functionality and integrity of the organism despite changes in the organism's environment, changes in diet and hydration, and many other variations and changes that influence the state of the organism. However, time, external effects and forces, and internal changes can degrade the finally turned, relatively low-entropy state of an organism, resulting in degradation of the homeostatic processes that maintain the integrity of the organism. For example, as an organism ages, progressive degradation of structures and functionalities of the organism occurs due to the cumulative effects of radiation, ingestion of deleterious chemical substances, intake of bacterial and viral pathogens, mechanical insults and traumas, internal generation of reactive oxygen species, deterioration of component functions, and other such effects. The order and structure of the organism, at the molecular level, can degrade, as represented by somatic mutations, changes in the patterns of chemical modifications of DNA and the histone proteins associated with DNA in chromosomes, changes in the monomer sequences of biopolymers. The organism can degrade at higher levels, including changes in the function and dynamics of metabolic processes, changes in numbers and structures of cellular organelles, structures of organ components and organs, changes in electrical interconnections between neurons in the brain, changes in the percentage of functional to dysfunctional cells and other components of organs, increased concentrations of non-functional and deleterious small molecules, biopolymers, and biopolymer complexes, and other such degradative processes and phenomena. Thus, the phrase “health entropy” refers to a generally qualitative indication of the current functional state of homeostatic processes and molecular, structural, and operational order of a human being, with an increase in health entropy representing an increase in disorder and decrease in the function of homeostatic processes and decrease in resilience of the human organism. As a person's health entropy increases, the person may generally become more susceptible to disease, undesirable conditions, and experience greater rates of structural and operational deterioration. Health entropy may increase due to various types of degradative effects and agents, such as reactive oxygen species, due to the cumulative effects of operational errors, such as failures to precisely replicate DNA, transcribe DNA into RNA, and translate RNA into proteins, as well as in internal signaling errors, errors in modifying biopolymers, changes in epigenetics, and other such effects, but health entropy may also decrease due to changes in diet, exercise, activity, and other such variables. It is unsurprising that health entropy increases with age, and that the rate of increase also increases with age.

### An Overview of Currently Disclosed Methods and Systems

There are many deficiencies in current health metrics and health-data analysis with respect to providing a more quantitative assessment of the health entropy for individuals. One deficiency is that current health metrics are based primarily on the occurrences of discrete diseases and fail to take into consideration sub-clinical conditions. More importantly, current health metrics fail to take account of the potentially non-linear effects of multiple concurrent diseases and conditions on health entropy and entirely fail to attempt to quantify the health entropy associated with particular organ systems and organs. There is, therefore, a need for providing a more quantitative assessment of health entropy with greater resolution in order to facilitate research, drug development, biomarker development, public-health monitoring and other organizational health-related research and activities, to provide a basis for medical diagnosis, health monitoring, and development of treatments and therapies for individuals, and to provide health-entropy assessments to individuals to enable individuals to monitor their health, to monitor medical treatments, therapies, and other interventions, to effectively access healthcare based on an understanding of an individual's current health status, to evaluate advice and proposed treatments and therapies, and to make well-reasoned lifestyle adjustments, including dietary adjustments, and adjustments in exercise and activities.

Determining and providing quantitative assessment of health entropy is, however, a difficult and complex endeavor, requiring collection of large amounts of health data while protecting privacy, processing the collected data to develop computational models, and using individuals' health data and the developed computational models to generate quantitative health-entropy metrics that can be transmitted back to individuals, used to continuously update the computational models, and transmitted to medical professionals, medical centers and organizations, researchers, and research organizations in order to provide a basis for all of the above-mentioned tasks, operations, and activities. Determining and providing quantitative assessment of health entropy is impossible without automated systems dedicated to continuous, secure health-data collection and storage, periodic computational-model generation, automated health-entropy-metric generation, and secure transmission of generated health-entropy-metric data to individuals, professionals, and organizations.

As discussed above, health entropy is, currently, a largely qualitative assessment of the functional state of homeostatic processes and order in a human organism. The human organism is far too complex and dynamic in order to attempt to compute a quantitative entropy value such as those computed in classical thermodynamics or statistical mechanics. The human organism is also an open system that exists in a non-equilibrium state, for which determination of classical state-variable values, such as entropy, by classical thermodynamic approaches is not currently possible. An initial step in generating quantitative health-entry metrics is to provide a standard or basis for estimating or representing health-entropy-metric values that can be interpreted and understood by individuals, professionals, and organizations. In the currently disclosed methods and systems, biological age has been selected as an estimate for health entropy since, as discussed above, health entropy is known to increase with age as a result of cumulative degradative processes and effects.

FIG. 8 illustrates the relationship of chronological age to health entropy and the relationship of biological age to both health entropy and chronological age. Plot 802 shows the relationship of health entropy to chronological age, with health entropy plotted with respect to vertical axis 804 and chronological age plotted with respect to horizontal axis 806. In general, averaged over many individuals, health entropy decreases from birth 808 to late adolescence or early maturity 810 and then increases thereafter, with the rate of increase steepening significantly after the chronological age of 50 (812). However, as shown in plot 814, the relationship of health entropy to chronological age for a particular individual can vary substantially from the average relationship shown in plot 802. As shown in plot 814 for a particular individual, the individual's health entropy follows the initial curve 816 of the average health-entropy/chronological-age curve shown in plot 802 but then begins to steeply increase up to a local peak 818 at a point between 40 and 50 years of chronological age. This may be due to the occurrences of one or more diseases, deleterious habits such as smoking or substance abuse, or for other reasons. The individual's health entropy then decreases to a low point 820 before then steeply, but somewhat irregularly, increasing.

Plot 1822 illustrates a conceptual approach to determining an individual's biological age. In this approach, the horizontal axis 824 has been continuously rescaled in order that the shape of the health-entropy/age curve 826 corresponds to the shape of the health-entropy/age curve 812, computed as an average result over many individuals, shown in plot 802. The rescaling is nonlinear. For example, during the steep rise in health entropy for the individual between chronological ages 30 and 40, shown in plot 814, rescaling has compressed the corresponding interval 828 along the horizontal axis 824 of plot 822. During the relatively flat overall change in health entropy for the individual between the chronological ages of 45 through 60, rescaling has lengthened the corresponding interval 830 on horizontal axis 824 of plot 822. On the right-hand side of FIG. 8, rescaled horizontal axis 824 and linearly scaled horizontal axis 806 are aligned and plotted vertically 832 to show how biological age indicated on the rescaled axis compares to chronological age on the linearly scaled axis. For example, when the individual whose health-entropy/chronological-age relationship is plotted in plot 814 was 40 years old, his or her biological age was slightly over 50 years. This is, of course, a hypothetical and qualitative illustration of the meaning of the phrase “biological age,” using hypothetical curves, but well illustrates the fact that biological age is a type of nonlinear scaling with respect to chronological age that reflects the departures of aging in an individual from the average aging and thus can serve as a metric value for comparing the health entropy of an individual to a standard or expected health-entropy curve for a population of individuals. In other words, when the biological age of an individual can be estimated based on various different collected health data for the individual, then the biological age of the individual can be considered proportional to the individual's health entropy at each point in time and can serve as a relative quantitative metric for the health entropy with respect to a standard or expected health-entropy curve.

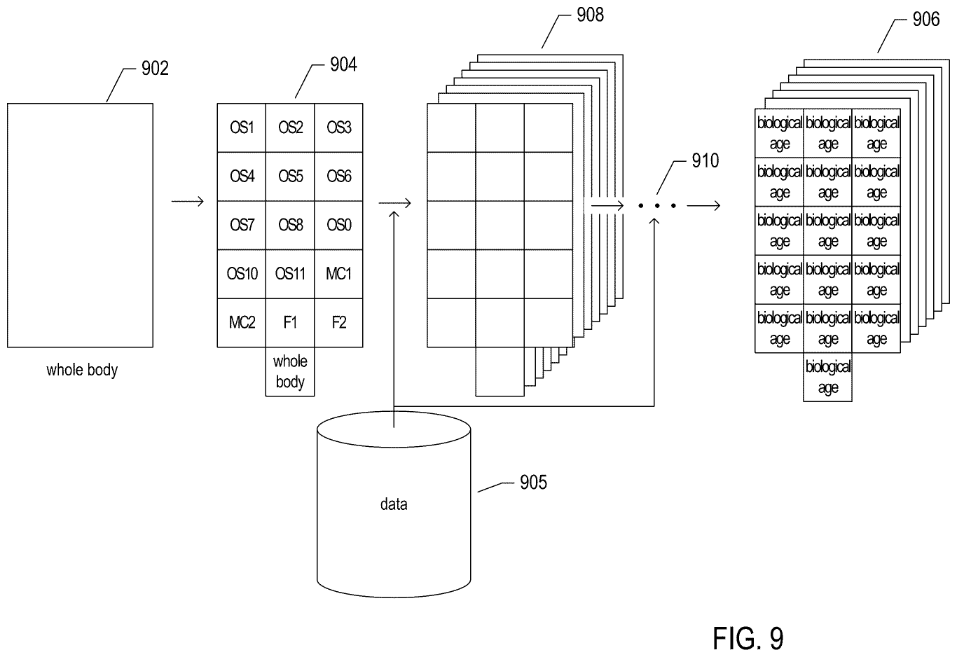

FIG. 9 illustrates one objective of the currently disclosed methods and systems. The currently disclosed methods and systems are intended to continuously collect large amounts of health data and information for many different people, to periodically develop and update computational models for determining health-entropy-metric values for individuals based on health data and other information submitted by the individuals, and to transmit the health-entropy-metric values to requesting individuals, professionals, and organizations to provide a basis for a variety of different types of medical-care, research and development, and individual tasks and decisions. Conceptually, the human body 902 is partitioned into a comprehensive collection of organ systems (“OSs”), conditions that span multiple organ systems or multi-organ-system conditions (“MCs”), and functionalities (“Fs”), in addition to being considered as a single, whole-body entity 904. Then, the currently disclosed system collects large amounts of health data and other information 905 and develops computational models for computing biological age, which represents a health-entropy-metric value, for each of the organ systems, multi-organ-system conditions, functionalities, and the whole body for each of multiple requesting individuals 906. During the process of computing biological ages, numerous additional metric values are computed, as represented by a set of additional metric values 908 and ellipsis 910. Three important concepts underlying this approach are: (1) use of biological age as a health-entropy-metric value; (2) partitioning the whole body into multiple organ systems, multi-organ-system conditions, and functionalities and computing biological ages for each of the multiple organ systems, multi-organ-system conditions, functionalities, and whole body; and (3) generating the health-entropy-metric values using various different types of health data and other data that represents not only clinical diseases, but also sub-clinical conditions and other phenomena and events that can contribute to the many different health-entropy-metric values. The above-mentioned partitioning is comprehensive in that, taken together, the organ systems and conditions span and well represent the whole body and thus provide a comprehensive picture of an individual's health entropy. Currently available health-entropy-related metrics fail to consider sub-clinical conditions and other such information and fail to provide metric values for organ systems, multi-organ-system conditions, and functionalities.

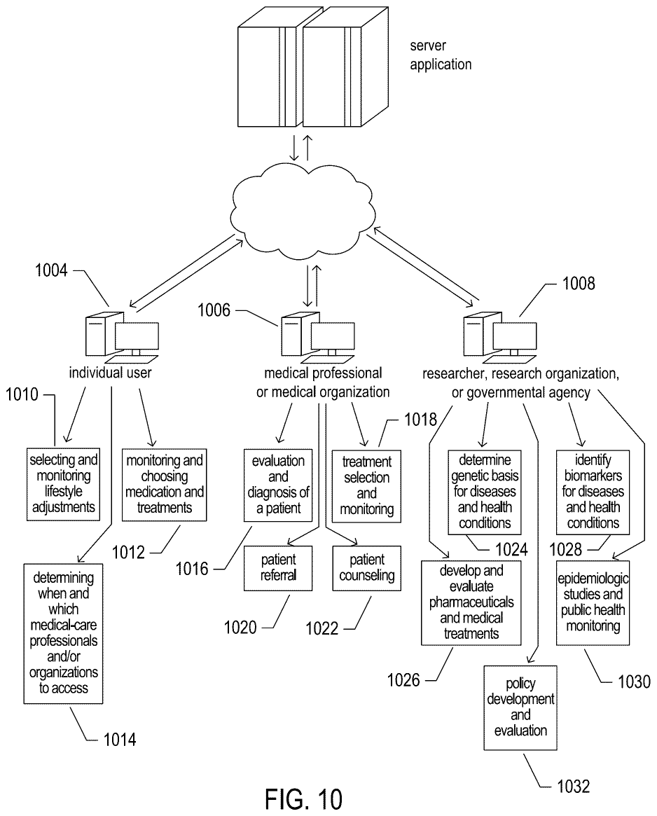

FIG. 10 provides a high-level illustration of the currently disclosed system. The system consists of one or more server applications running within one or more cloud-computing facilities or data centers 1002 that securely communicate with individual-user computers 1004, medical-professional and medical-organization computers 1006 and researcher and research-organization computers 1008. The server application is generally implemented by a collection of virtual machines within one or more cloud-computing facilities or data centers, often including functional layers of virtual-machine components, such as back-end servers, data-storage components. A variety of different implementations is possible. A webserver can collect information from users and display information to users via a web browser running on user computers. Alternatively, the user computers can each execute a client-side application that communicates with the server application running within one or more cloud-computing facilities and/or data centers. The server application receives health data and other information from individual users, medical professionals and researchers, and medical organizations and transmits health-entropy-metric values and other information generated from the collected health data and other information as well as from the computational models used to generate the metric values to individual users, medical professionals and medical organizations, and researchers and research organizations. Individual users can use the health-entropy-metric values returned to them for many different purposes, including selecting and monitoring lifestyle adjustments 1010, monitoring and selecting medication and treatments 1012, and determining when and which medical-care professionals and/or organizations to access for particular needs 1014. Medical professionals and medical organizations can use health-entropy-metric values for individuals or various types of derived information from the computational models and stored health-entropy-metric values for multiple of individuals for many different purposes, including evaluating individuals' health and diagnosing individuals' conditions and diseases 1016, selecting treatments and monitoring treatments 1018, selecting professionals or organizations for patient referrals 1020, and for various types of patient counseling 1022. Researchers and research organizations can use the various types of derived information from the computational models and stored health-entropy-metric values for multiple of individuals for many different purposes, including determining the genetic basis for particular diseases and health conditions 1024, developing and evaluating pharmaceuticals treatments and disease-prevention strategies 1026, identifying biomarkers for disease and health-condition prediction and prevention, diagnosing and detecting various types of diseases and health conditions 1028, conducting epidemiological studies and public-health monitoring 1030, and developing and evaluating various types of health-related policies 1032. Of course, there may be many additional uses for information derived from the computational models developed to compute health-entropy-metric values and the health-entropy-metric values themselves. It is important that metric values and other derived results be anonymized before being transmitted to research organizations and to any organization or individual not currently treating or counseling the individual or individuals from whom the data was collected to generate the metric values and other derived results.

There are several important aspects of the currently disclosed methods and systems that need to be considered. First, the vast majority of the computational overheads associated with the currently disclosed system arise from data collection, computational-model generation, and transmission of metric data and derived results to user individuals and organizations. Computing the health-entropy-metric values represents a relatively small portion of the computational overheads. Second, in the following discussion, mathematical notation is used to describe many of the computational tasks carried out by the currently disclosed system, but mathematical notation is simply a descriptive language that is more concise and precise than attempting to describe the large sequences of computer instructions that implement the control logic of the currently disclosed system and the order in which they are executed within the virtual machines resident in data centers and cloud-computing facilities. The currently disclosed system does not generate mathematical notation or expressions nor does the currently disclosed system carry out symbolic mathematical operations. Instead, the currently disclosed system communicates with users, serves webpages, in the case of a webserver implementation, ensures privacy and confidentiality of user data, computationally generates models and model parameters that can be used to compute health-entropy-metric values from health data and other information, and computes, for requesting individuals and organizations, health-entropy-metric values for each of multiple individuals from various types of health data and other information submitted to the system by the multiple individuals. Surprisingly, many fail to understand that mathematical expressions are simply a language, just as are programming languages and natural languages. When mathematical expressions are used to describe computer-implemented systems, they describe the execution of computer instructions to implement processes just as programming languages describe the execution of computer instructions to implement processes.

### An Overview of Probability and Statistics

FIGS. 11-25 provide an overview of probability and statistics that may be useful to a reader for understanding the computations carried out by the currently disclosed methods and systems. This section is relatively brief and intended more to facilitate recollection than to teach. Full discussions can be found in many different textbooks. Also, again, the mathematical expressions provided in FIGS. 11-25 are simply descriptions of processes. The currently disclosed methods and systems do not generate mathematical expressions and do not carry out symbolic mathematical operations.

An initial set of expressions 1102 at the top of FIG. 11 illustrates the average, variance, and standard-deviation metrics commonly encountered in probability and statistics. A set of measurements m is shown to comprise a set of 10 floating-point values 1104. The number of measurements in the set is represented by the notation 1105 and the sum of the measurements is represented by expression 1106. The average measurement, as indicated by expression 1107, is the sum of the measurements divided by the number of measurements. An estimate for the variance, as indicated by expression 1108, is the sum of the squared differences between the individual measurements and the measurement average divided by one less than the number of measurements. The standard deviation, as indicated by expression 1109, is the square root of the variance.

Diagram 1110 represents a sample space S. A sample space is an abstract space that includes a set of simple events that occur during an experiment. Each simple event is represented in the diagram by a numerically labeled circle. Subsets of the sample space, or sets of simple events, such as set 1111, also represent events. A sample space is discrete when the number of simple events is finite or countable but is continuous when the number of simple events is infinite or uncountable. Experiments are conducted on the sample space, with each experiment resulting in a single simple event. The probability of the occurrence of events during experiments is the subject matter of the field of probability. Examples of sample spaces include the possible outcomes of a roll of a pair of dice, the number of defective parts in each batch of n parts, and the possible five-card hands dealt in a poker game, with corresponding experiments comprising a roll of the pair of dice, examination of a batch of parts to detect the number of defective parts, and dealing a five-card hand from a deck of cards. When the sample space includes more than a single simple event, which is almost always the case and the only interesting case, each simple event e is associated with a probability P(e) with a real-number value greater than 0 and less than 1 (1112), with the sum of the probabilities of all of the simple events equal to 1 (1113). The probability of an event that is a set of simple events is equal to the sum of the probabilities of the simple events that comprise the event 1114. When such an event is the null set, the probability of the event is 0 and, otherwise, is greater than 0 (1115). Many probability expressions are based on set operations which are illustrated in the lower portion 1116 of FIG. 11 and which include the union operation 1117, the intersection operation 1118, the subset operation 1119, the superset operation 1120, and the complement operation 1121.

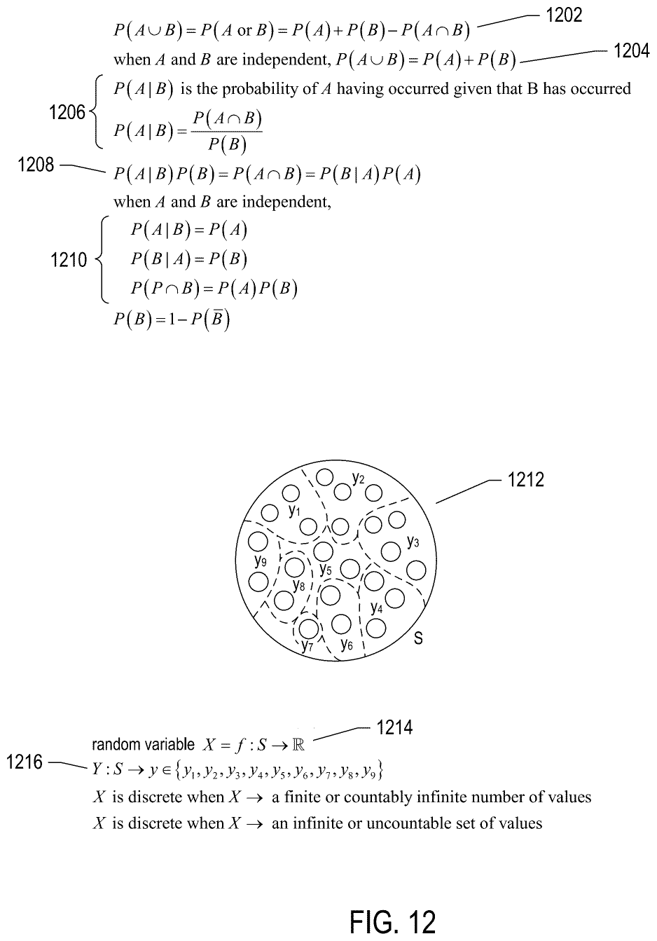

There are a number of well-known probability expressions. These are shown in the upper portion of FIG. 12. The probability of the occurrence of either of two events A and B, or the probability of event A OR event B, is equal to the sum of the probabilities of each of the events minus the probability of event A AND event B 1202. When two events A and B are independent, the probability of A OR B is equal to the sum of the probabilities of the two events 1204. The conditional probability of event A given the occurrence of event B is the probability of A AND B divided by the probability of B 1206. The conditional probability of A given the occurrence of B times the probability of B is equal to the probability of A AND B which is, in turn, equal to the conditional probability of B given the occurrence of A times the probability of A 1208. Expressions 1210 are true when events A and B are independent. The probability of B is equal to 1 minus the probability of the complement of B.

Diagram 1212 in FIG. 12 shows the partitioning of a sample space S into nine partitions or subsets, each of which includes one or more simple events. Each of the partitions, or subsets, is associated with a unique real number designated as yᵢ, where i is an integer in the range [1,9]. A random variable X is a function that maps partitions of a sample space to real numbers 1214. Thus, for example, the random variable Y 1216 is a function with the domain equal to the sample space S that returns one of the real numbers yᵢ. In other words, a random variable is a map of events to real numbers. A random variable is discrete when it maps a sample space to a finite or countably infinite number of real values and discontinuous when it maps a sample space to an infinite or uncountable set of values.

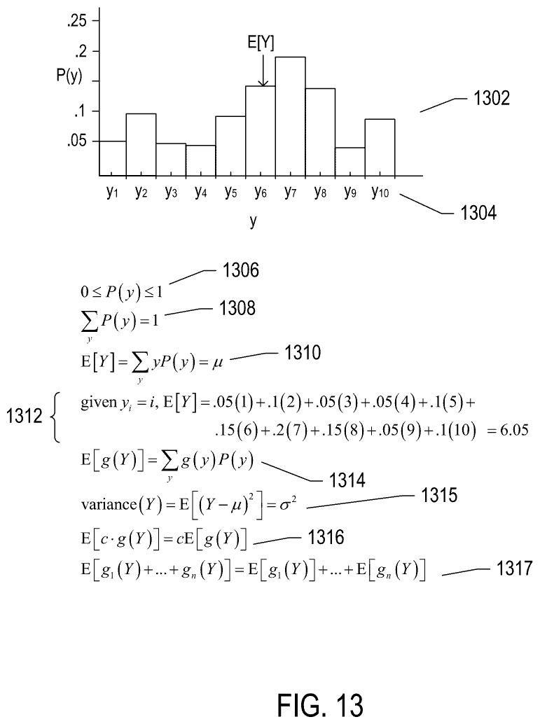

Diagram 1302 at the top of FIG. 13 shows a discrete probability distribution for a hypothetical random variable Y that takes on values y₁, y₂, . . . , y₁₀, plotted along the horizontal axis 1304. The heights of the rectangular columns above each possible value of the random variable Y indicate the probability of the occurrence of that value in a single experiment. The ordered sequence of probabilities corresponding to the ordered sequence of possible values comprises a discrete probability distribution. The probability of the random variable returning any particular value y is greater than or equal to 0 and less than or equal to 1 1306 and the sum of the probabilities in the discrete probability distribution is 1 1308. The expected value of Y is computed as the sum of each possible value times the probability of that value and is the mean u of the random variable 1310. An example calculation of the expected value of a random variable Y is shown in expression 1312 in FIG. 13. The expected value of any function of the random variable Y is equal to the sum of the products of the function of each possible value of the random variable and the probability of the value of the random variable 1314. Expression 1315 shows the variance of the random variable to be the expected value of the square of Y−μ. The expected value of a constant times a function of the random variable is equal to the constant times expected value of the function of the random variable 1316. The expected value of a sum of functions of the random variable is equal to the sum of the expected values of the functions 1317.

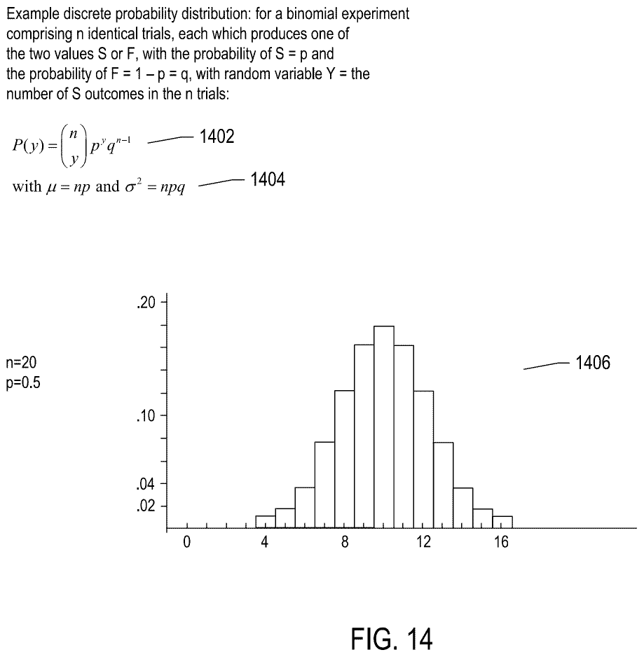

FIG. 14 shows an example discrete probability distribution referred to as the “binomial distribution.” It is the probability distribution for a binomial experiment comprising n different identical trials, each producing one of two possible values S and F representing success and failure. The probability of S is a real number p in the range (0, 1) for non-trivial cases and the probability of F is 1−p=q. The random variable Y is equal to the number of S′ outcomes in the n trials. The expression 1402 indicates how the probability of any particular value y of the random variable Y can be computed, namely the number of different ways to select y items from n items times p to the y^{th} power times q to the n−1 power. The mean value for the random variable Y is equal to np and the variance for the random variable Y is equal to npq 1404. Plot 1406 shows the discrete binomial probability distribution for n=20 and p=0.5.

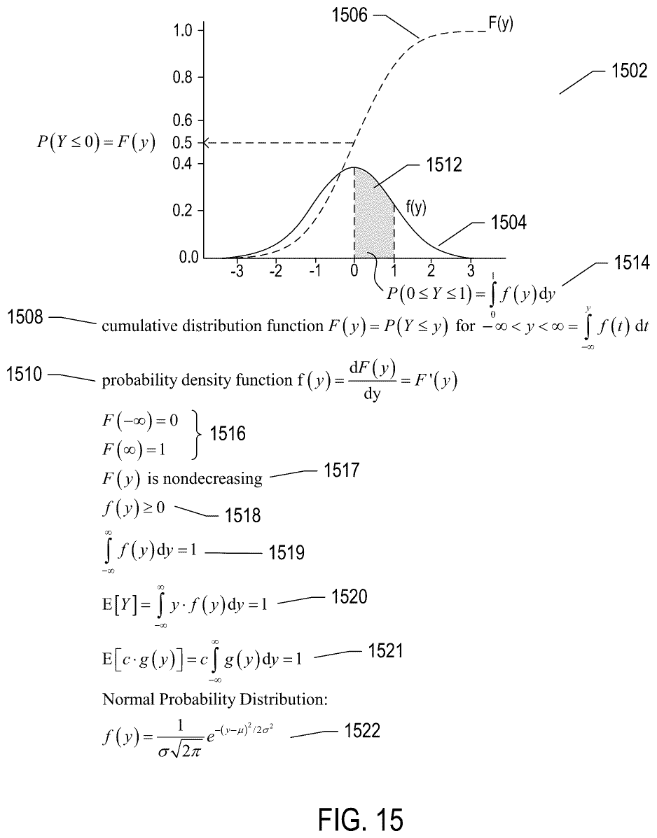

FIG. 15 illustrates probably the most often encountered continuous probability density function known as the “normal probability density function” along with the related cumulative normal distribution function. At the top of FIG. 15, a diagram 1502 is shown which includes the normal probability density function ƒ(y) 1504 and the cumulative normal distribution function F(y) 1506. As shown in expression 1508, the cumulative normal distribution function takes a real-number value y as an argument and returns the probability that the random variable Y returns a value less than or equal to y and can be calculated as the integral of the normal probability density function from minus infinity to y. As shown in expression 1510, the normal probability density function is the derivative of the cumulative normal distribution function. As shown in diagram 1502, the probability that the random variable Y lies between two values, such as 0 and 1, is the area under the normal probability density function between the two values, illustrated by the shaded region 1512 and expression 1514. The cumulative normal distribution function necessarily has the value 0 at minus infinity and the value 1 at plus infinity, as indicated by expressions 1516. A cumulative distribution function is nondecreasing 1517 and the probability density function returns values greater than or equal to 0 (1518). The area under the probability density function curve is 1 (1519) and the expected value of random variable Y is found by integrating the product of y times the probability density function of y, as shown in expression 1520. The expected value of a function of the random variable Y multiplied by a constant is the product of the constant times the integral of the function of y times the probability density function of y 1521. Expression 1522 shows the analytical form for the normal probability density function, where μ and σ are the mean and variance for Y.

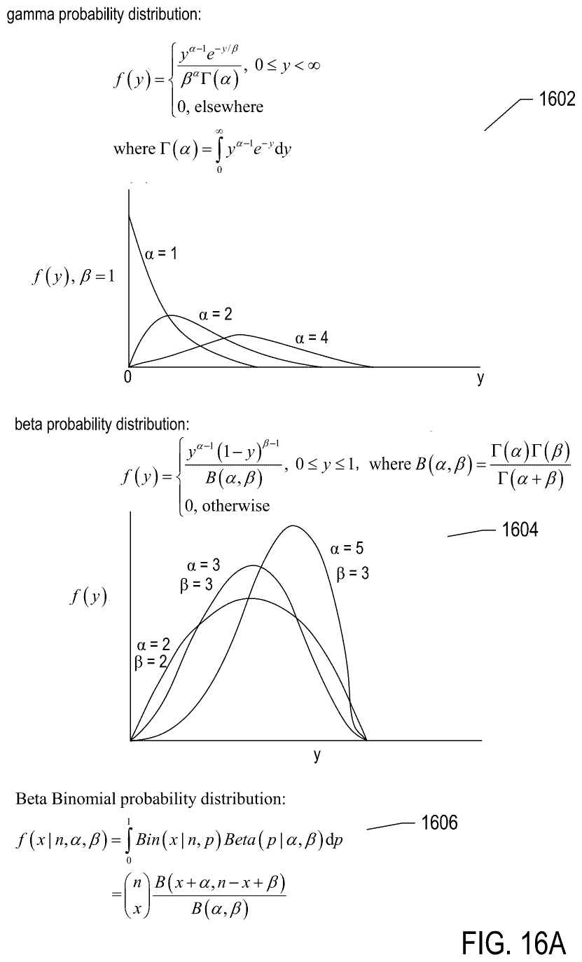

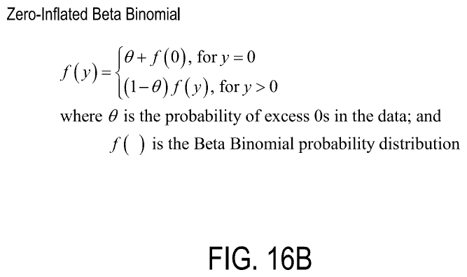

FIG. 16A illustrates three additional continuous probability density functions using the same notational conventions used in FIG. 15. These include the Gamma probability distribution 1602, the Beta probability distribution 1604, and the Beta Binomial probability distribution 1606. The analytical expressions for these distributions include parameters such as α, β, n, and p. FIG. 16B illustrates the Zero-Inflated Beta Binomial distribution. This distribution is used for data that has an excess of 0 values than would be expected were the data to be distributed according to the Beta Binomial distribution, but that otherwise is distributed according to the Beta Binomial distribution. The Zero-Inflated Beta Binomial distribution is described by two expressions, the first expression 1610 is for the probability of y=0 and the second expression 1612 is for y>0.

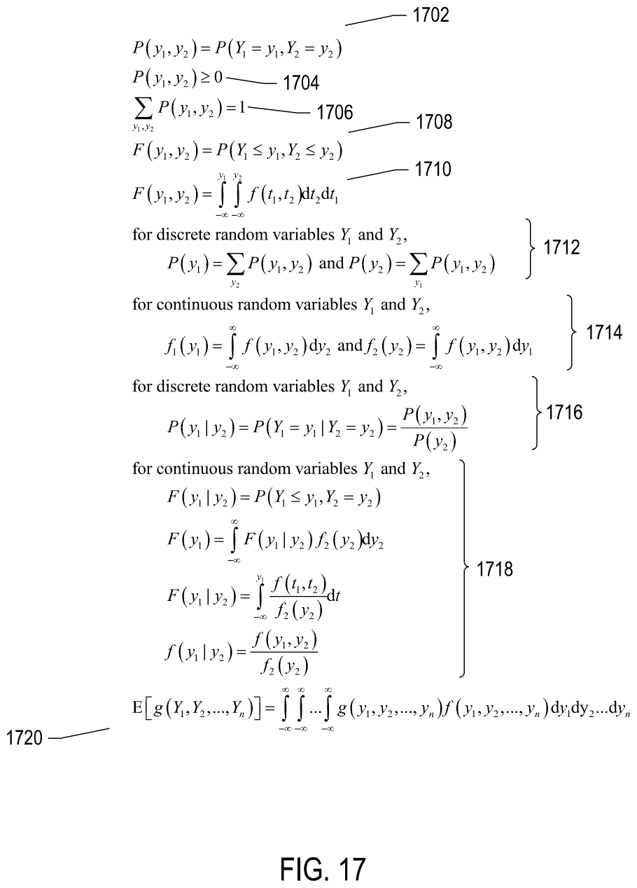

FIG. 17 illustrates properties of bivariate and multi-variate cumulative distribution functions and probability density functions. As shown in expression 1702, the probability that two random variables Y₁ and Y₂ have two particular values is expressed as P(y₁, y₂), where y₁ and y₂ are the two particular values. Such two-random-variable probabilities are associated with bivariate probability density functions and cumulative distributions. Of course, the probability that two random variables have two particular values is necessarily greater than or equal to 0 (1704) and the sum of the probabilities for all combinations of the possible values of the two random variables is equal to 1 (1706). Expression 1708 defines the bivariate cumulative probability distribution and expression 1710 illustrates determination of the bivariate cumulative probability distribution via integration of the corresponding bivariate probability density function. Expressions 1712 illustrates computation of the marginal probabilities for discrete bivariate probability density functions. Expression 1714 similarly indicates computation of the marginal probabilities for continuous bivariate probability density functions. A marginal probability density function is a univariate probability density function for one of the two random variables associated with a bivariate probability density function. Expressions 1716 illustrates computation of the conditional probabilities for discrete bivariate probability density functions and expressions 1718 illustrates conditional cumulative probability distributions and probability density functions for two continuous random variables. Finally, expression 1720 illustrates computation of the expected value of a function of multiple random variables.

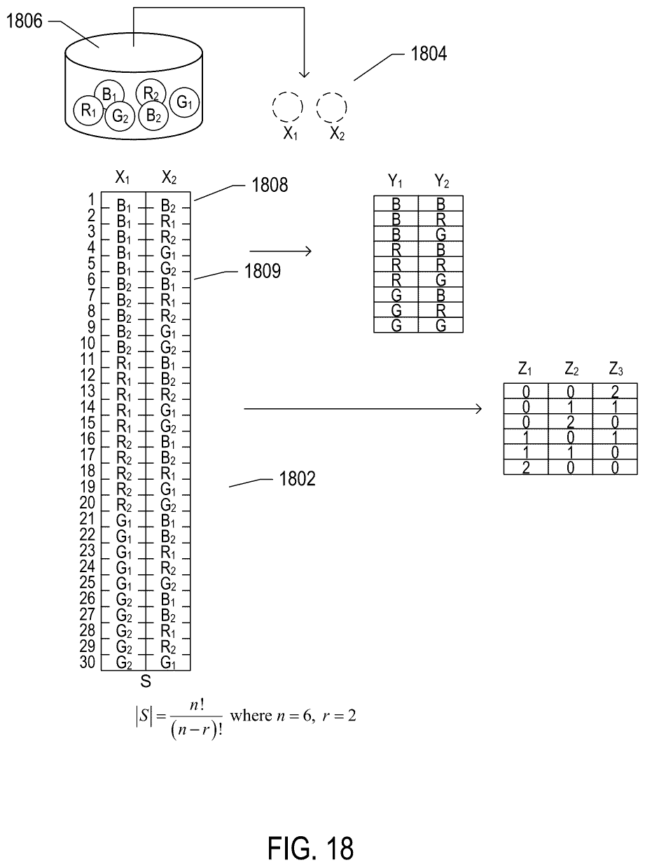

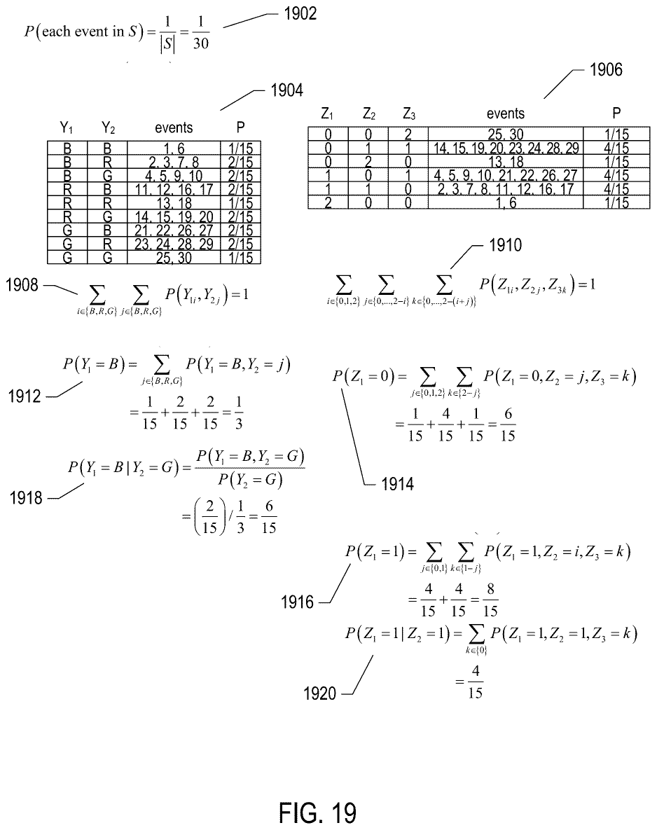

FIGS. 18-19 provide a simple example of a sample space, two different sets of random variables defined over the sample space, and the computation of probabilities and conditional probabilities for the random variables. The sample space 1802 contains all possible ways of randomly selecting two labeled balls 1804 from a bin 1806 containing two labeled balls of each of the three colors blue, green, and red. Note that the order of the selections is considered so that, for example, there are two sample points 1808-1809 corresponding to selecting two blue balls from the bin. Two random variables are defined to represent, respectively, the selection of the first ball of a particular color and the selection of the second ball of a particular color. The three random variables Z₁, Z₂, Z₃ are defined to represent, respectively, the number of blue balls selected, the number of red balls selected, and the number of green balls selected. This illustrates how different sets of random variables can be defined over a given sample space. As indicated by expression 1902 at the top of FIG. 19, each event in the sample space is considered to have the same probability of occurrence, namely 1/30. Table 1904 shows the probabilities for all possible values of the two random variables Y₁ and Y₂, the probabilities associated with these values, and the sample-space events associated with the values. Table 1906 similarly shows the probabilities for all possible values of the three random variables Z₁, Z₂, Z₃, the probabilities associated with these values, and the sample-space events associated with the values. Expressions 1908 and 1910 illustrate computation of the sums of the probabilities of the possible values for the random variables Y₁ and Y₂ and the random variables Z₁, Z₂, Z₃, respectively, similar to expression 1706 in FIG. 17. Expressions 1912, 1914, and 1916 illustrate computation of marginal probabilities for the two sets of random variables and expressions 1918 and 1920 illustrate computation of conditional probabilities.

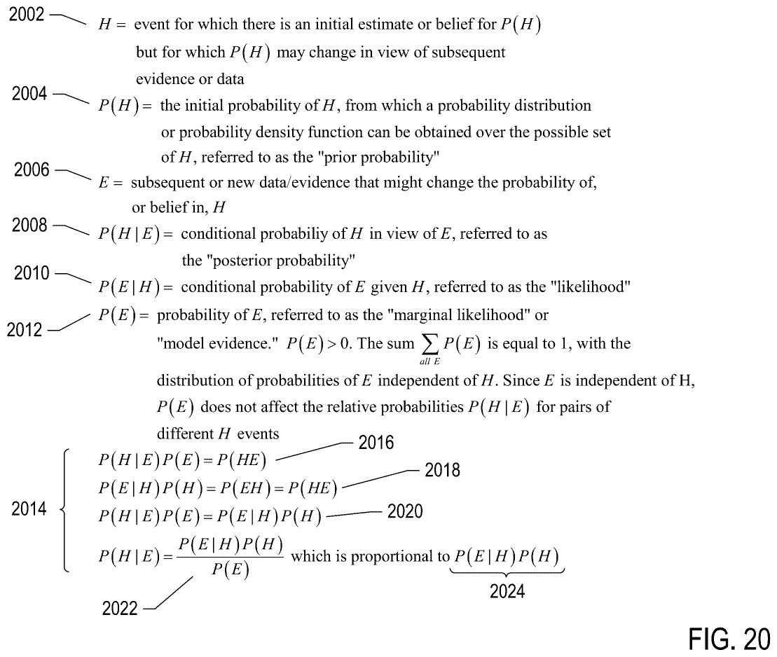

The currently disclosed methods and systems rely on Bayesian inference for generating computational models for computing health-entropy-metric values and other values. FIGS. 20-23 illustrate Bayesian inference and Bayesian-inference-based regression. In the top portion of FIG. 20, each main part of the expression for Baye's rule is described along with the notation for the parts. H represents an event or a set of events for which there is an initial estimate of, or believe in, the probability of the occurrence of H but for which the probability of occurrence may change in view of additional evidence or data 2002. P(H) is the initial estimate of, or believe in, H and can also be considered either as the probability of a particular event or as the probability density function for all possible events in a set of events H 2004. E represents the subsequent data that might result in a change or adjustment of the probability of, or belief in, the event H or the set of events H 2006. P(H|E) is the conditional probability of H in view of the additional evidence E, and is referred to as the “posterior probability” 2008. It is the result of Bayesian inference. P(E|H) is the conditional probability of H in view of E, and is referred to as the “likelihood” 2010. Finally, P(E) is the probability of the additional evidence or data E, referred to as the “marginal likelihood” or “model evidence” 2012. The probability of the additional evidence is greater than 0 and the sum of the probabilities of all possible additional evidence is equal to 1. The probability of the evidence is independent of the probability of H.

Expressions 2014 in the lower portion of FIG. 20 illustrate a derivation of Baye's rule. Expressions 2016 and 2018 follow from expressions 1206 and 1208 in FIG. 12, discussed above. Expression 2020 follows from expressions 2016 and 2018. Multiplying both sides of expression 2020 by 1/P(E) produces an expression for Baye's rule 2022. Since P(E) is independent from P(H), and is simply a constant normalizing factor for a given E, Baye's rule is often shown as a proportionality 2024. In other words, when thinking of P(E|H) and P(H) as probability density functions, their sum is also a probability density function, but one that is not normalized meaning that the area under the curve is not equal to 1. Dividing this normalized probability distribution by P(E) generates a normalized probability distribution.

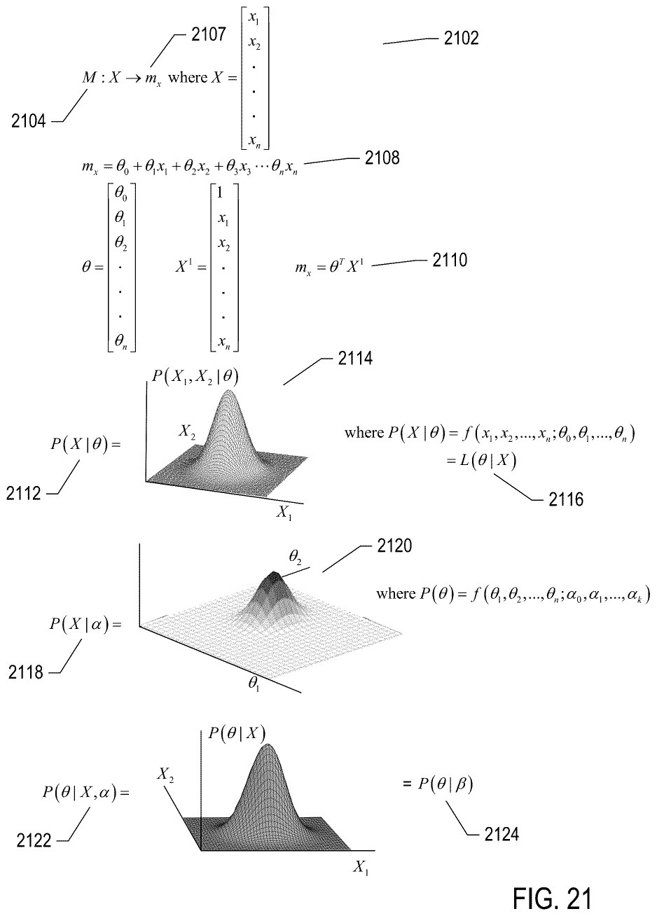

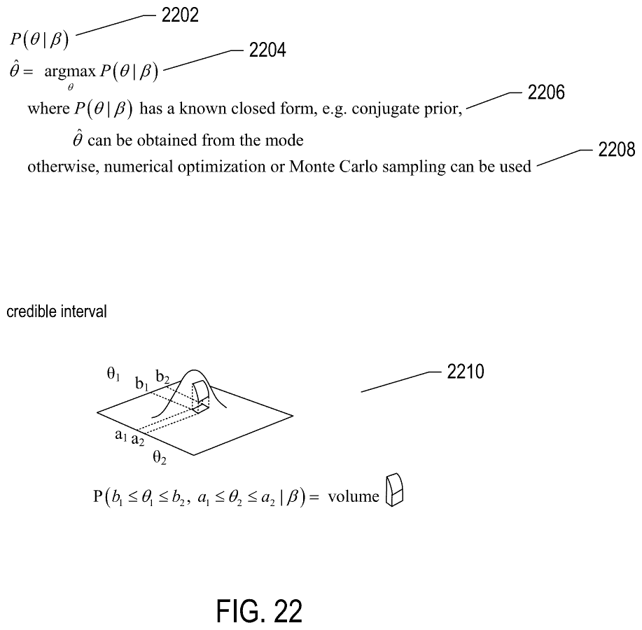

The currently disclosed methods and systems employed Bayesian inference in order to generate the computational models needed for determining health-entropy-metric values. FIGS. 21-22 illustrate components of the computational-model-generation process. As shown in expressions 2102 at the top of FIG. 21, a computational model M can be thought of as a function 2104 that accepts a vector of data values X 2106 and returns a metric value 2107. In many cases, the metric value is computed from the data-value elements of the vector X as a sum of terms, each comprising a parameter θ multiplied by a data-value element 2108. This can be expressed as a dot product of the two vectors X and θ 2110. In order to determine the values of the parameters, a Bayesian-inference-based regression is carried out over a data set. The likelihood 2112 for the Bayesian inference is a multi-variate probability distribution represented by bivariate probability distribution 2114 since higher-dimensional probability distributions cannot be graphically represented. The likelihood is often expressed as 2116 to indicate that it is considered to be a function of the parameters when used in Bayesian inference. The prior for the Bayesian inference 2118 is also a probability distribution, again represented by a bivariate distribution 2120 since higher-dimensional distributions cannot be graphically illustrated. The prior can be thought of as a conditional probability distribution of the parameters given a vector of distribution-parameter values a for the probability density function. The posterior 2122 is also a conditional probability distribution, namely the probability of the parameters given the data and the posterior distribution-parameter values a for the prior probability density function. In certain cases, the posterior conditional probability distribution is a different probability density function with different parameters β. In certain cases, both the prior and posterior conditional probability distributions have the same functional form and differ only in the parameters. In other cases, the posterior conditional probability distribution is a different type of distribution than the prior conditional probability distribution. In many cases, the posterior conditional probability distribution is approximated by various types of approximation methods, including sampling-based methods and is not associated with a closed-form expression for the probability density function. The notation 2124 is used, in the current illustration, for the posterior conditional probability distribution to indicate one of any of the above-mentioned types, either a conditional probability distribution of the closed-form expression or a conditional probability distribution approximated by some type of approximation methods. With the posterior conditional probability distribution in hand, shown in expression 2202 at the top of FIG. 22, a new set of parameter values can be estimated as an optimization problem 2204 in which the parameter values that maximize the probabilities of the parameter values are selected. For example, when the posterior conditional probability distribution has an unknown closed form, the estimation for the parameters can be obtained from the mode or mean statistic for the distribution 2206. Otherwise, numerical optimization or Markov Chain Monte Carlo sampling can be used to estimate new parameter values 2208. Because Bayesian inference returns a probability density function as the posterior, it is relatively straightforward to determine the probability that the new parameter values determined by Bayesian inference fall within particular intervals 2210. Therefore, it is straightforward to determine a credible interval, analogous to a confidence interval, for the parameter values.

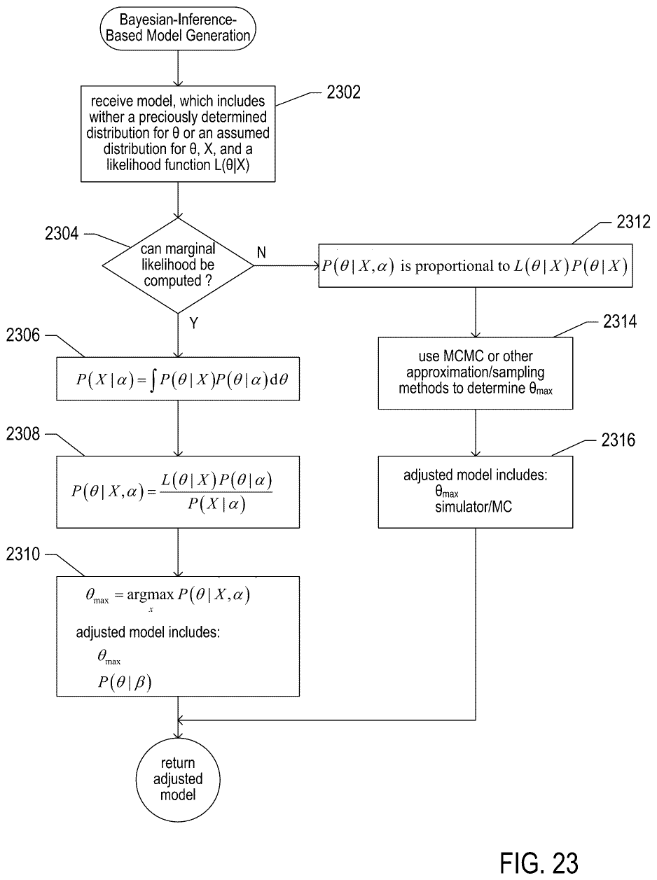

FIG. 23 provides a control-flow diagram to illustrate Bayesian-inference-based model generation. In step 2302, a model is received along with either a previously determined prior distribution for the model parameters or an assumed prior distribution for the model parameters. In addition, a data vector X is received along with a likelihood function L(θ, X). In conditional step 2304, a determination is made as to whether the marginal likelihood can be computed. If so, then the marginal likelihood is computed in step 2306, the posterior distribution is computed in step 2308, and new values for the model parameters are computed in step 2310. Otherwise, when the marginal likelihood cannot be computed, a relative posterior distribution is computed in step 2312, the new parameter values are computed in step 2314 using sampling or approximation methods, and the new model including the new parameters is generated in step 2316. This process can be sequentially repeated for a sequence of data vectors, with the model parameters adjusted following processing of each data vector by the model-generation process illustrated in FIG. 23. The posterior generated in one iteration of the sequential application of the method shown in FIG. 23 is used as the prior for the next generation of the sequential application of the method shown in FIG. 23.

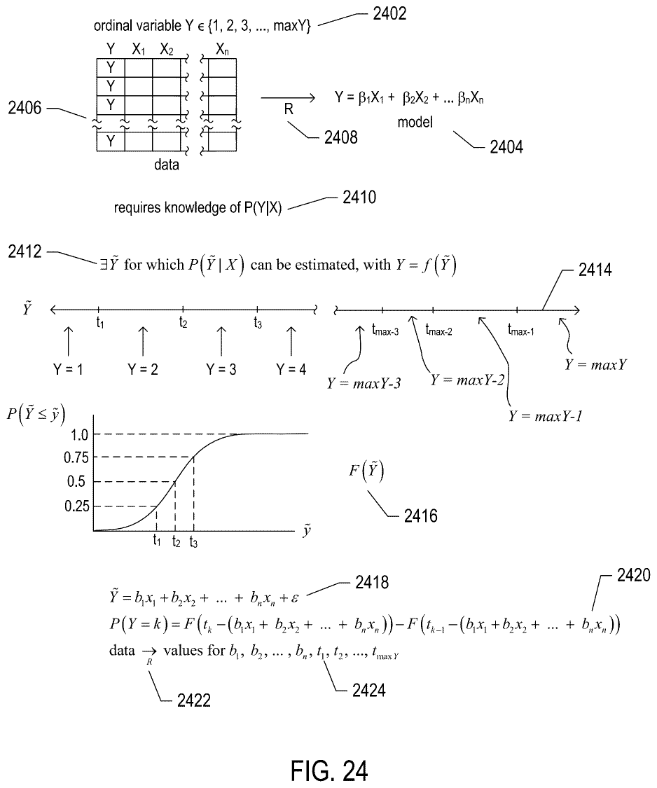

FIG. 24 illustrates Bayesian ordinal regression with a cumulative distribution. This technique is used for models that predict the value of an ordinal random variable. It can be the case that the ordinal values are not equally distant from one another in probability, as a result of which Bayesian-inference-based regression may fail to properly estimate the model parameters. As shown in expression 2402 at the top of FIG. 24, the example shown in FIG. 24 involves generating a model for an ordinal random variable Y which can have integer values between 1 and some maximum integer value max Y. Of course, the range of possible values may differ for different ordinal random variables. It is desired to generate a model 2404 from a set of data values 2406 using Bayesian-inference-based regression 2408. Model generation requires some kind of notion of the probability of predicted values of Y given a data vector 2410, but in many cases, the probability distances between successive possible values of Y are not known. The Bayesian-ordinal-regression technique assumes the existence of an unknown latent random variable 2412 from which the probability of given data X can be estimated and for which a function ƒ( ) returns a corresponding value of ordinal random variable Y. The technique involves placing cut points tᵢ along the real-number line segment representing the possible values of 2414 in order to generate cut-point-delimited intervals to correspond to ordinal values of ordinal random variable Y such that the probabilities for each possible value of ordinal random variable Y are equal. Diagram 2416 illustrates the cumulative distribution for along with the cut points t₁, t₂, and t₃ which divide the real-number-line segment along which possible values of are plotted into four equal-probability regions corresponding to four possible values of Y (assuming Y is 4-valued). The model for 2418 is then used to generate a model 2420 for the probability density function for ordinal random variable Y that includes the parameters of model 2418 along with parameters corresponding to the cut points. Bayesian-inference-based regression 2422 can then be used to estimate values for the parameters 2424 of a model for random variable Y. The model for random variable Y generates real-number values rather than ordinal values.

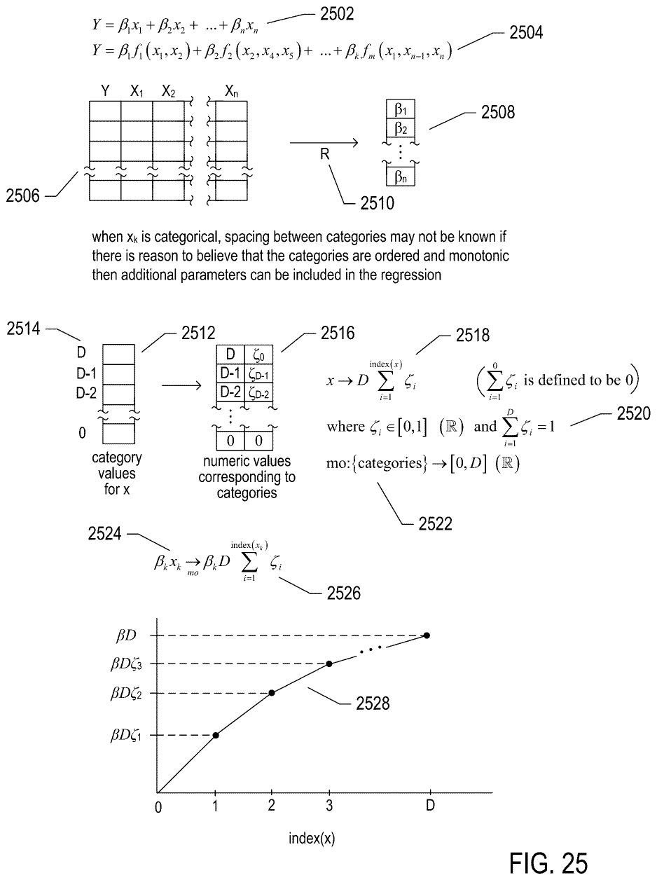

The problem with unequally spaced ordinal random variables discussed above with reference to FIG. 24 also extends to ordinal or categorical model predictors. FIG. 25 illustrates a “monotonic effect” method that, similar to the method discussed above with reference to FIG. 24, introduces additional model parameters to handle unknown spacing between category values of each categorical model predictor that is believed to represent ordered and monotonic categories. This technique can be used for models such as model 2502, where each predictor xᵢ is multiplied by a parameter βi to generate the i^{th} term and the terms are then added to generate a predicted value for a random variable Y, as well as model such as model 2504, where the parameters βi multiply values of the functions of one or more of the predictors. Again, the intent is to use data 2506 to generate parameter values 2508 via Bayesian-inference-based regression 2510. The technique begins with the set of category values for each categorical predictor 2512. These category values are indexed 2514 from 0 to D=the number of categories+1. Then, the indices are associated with variables ζi 2516. A predictor with a known category value can then be represented as the sum of ζi for i=1 up to the index of the category value times the maximum index D 2518. In the case that the index of the category value is 0, the sum of ζi for i=1 up to the index of the category value is defined to be 0. Each ζi represents the percentage of the full range of category values associated with a predictor attributable to the category value indexed by i. Therefore, each ζi has a value between 0 and 1 and the sum of all of the ζi for a predictor is equal to 1 (2520). Thus, the monotonic-effect transform 2522 transforms categories for a predictor to real numbers in the range of zero to D. Thus, a term corresponding to a categorical predictor in the model 2524 is transformed into the form 2526 with additional parameters comprising the ζi. Thus, the possible term values for the possible indices of a predictor value follow a monotonically increasing curve 2528 of straight-line segments.

### The Currently Disclosed Methods and Systems

The currently disclosed methods and systems, as discussed above, include an automated system implemented by one or more server applications and/or web servers running within one or more data centers and/or cloud-computing facilities that exchange data and information with computers and other processor-controlled devices used by individuals, including individuals providing health information and other information in exchange for receiving health-entropy-metric value, used by medical professionals and healthcare organizations, and used by researchers and research organizations. Control-flow diagrams that illustrate an implementation of the currently disclosed methods and systems are provided in FIGS. 26-34B.

FIG. 26 provides a control-flow diagram for the server application. In step 2602, the server application initializes or re-initializes various data structures, communications connections, one or more databases, and other computational infrastructure in preparation for handling requests received from users and transmitting responses to the users. In certain cases, the server application is initialized with computational models generated from archived health data and other information, and the computational models are continuously refined and updated based on subsequently received health data and other information. In alternative implementations, the initial computational models may be based on various types of assumptions and beliefs, and the server application may need to receive a threshold amount of health data and other information from users before model parameters are sufficiently adjusted and refined to provide sufficiently accurate health-entropy-metric values. It should be noted that the currently disclosed system does not simply apply static models to input data, but instead periodically generates and refines computational models during operation. The computational models are not formulaic or predefined, but evolve, over time, depending on the data that the system receives. This continuous refinement provides real-time information to researchers that they would be generally unable to obtain as precompiled data sets from information sources. As mentioned above, all data exchanges are secured by encryption and other means to ensure that individuals' health data cannot be intercepted by malicious parties and researchers generally receive only anonymized data that would not allow them to infer to which individuals the data corresponds.

In step 2604, the server application waits for a next event. Events include reception of requests from users, various types of administrative requests and tasks that need to be performed, and internally generated events, such as completion of tasks by back-end processing components. Following the occurrence of a next event, the server application calls a particular handler routine depending on the type of event that has occurred. When the event is a login request from a user, as determined in step 2606, a login-request handler is called in step 2608. When the event is a logout request, as determined in step 2610, a logout-request handler is called in step 2612. When the next event is reception of data from a user, as determined in step 2614, a new-data handler is called in step 2616. When the next request is a request for personal health-entropy-metric values and other computed values, as determined in step 2618, a computed-value-request handler is called in step 2620. Ellipses 2622-2623 indicate that many other types of events may occur and are handled by calling corresponding handler routines. A final default handler 2624 handles any rare or unexpected events. When at least one other event has been queued for handling, as determined in step 2626, the next event is dequeued in step 2628 and control returns to step 2606 to handle the next event. Otherwise, control returns to step 2604 where the server application waits for the occurrence of a next event. In the interest of conciseness and focusing on relevant portions of the currently disclosed system to which claims are directed, only the new-data handler 2616 and the computed-values-request handler 2620 are further illustrated and discussed in the current document.

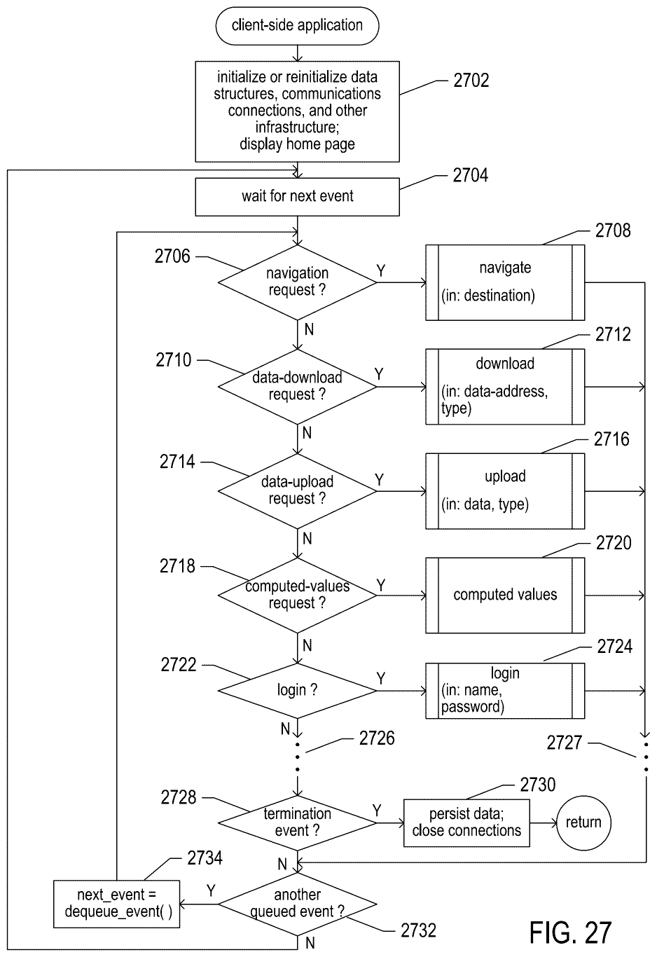

FIG. 27 provides a control-flow diagram for the client-side application that runs on user computers in certain implementations of the currently disclosed system. In step 2702, the client-side application initializes or re-initializes data structures, communications connections and other such computational infrastructure and, in certain implementations, displays a home page or landing page to the user. Then, in step 2704, the client-side application waits for the occurrence of a next event. When the next event is a navigation request, as determined in step 2706, a navigation-handler is called in step 2708. When the next event is a data-download request, as determined in step 2710, a download handler is called in step 2712. When the next event is a data-upload request, as determined in step 2714, an upload-handler is called in step 2760. When the next event is a computed-values request, as determined in step 2718, a computed-values handler is called in step 2720. When the next event is a login request, as determined in step 2722, a login handler is called in step 2724. Ellipses 2726-2727 indicate that many other types of events may occur and are handled in similar fashion by calling corresponding handlers. When the next event is a termination event, as determined in step 2728, the client-site application persists the memory-resident data that needs to be stored and closes communications connections, in step 2730, before terminating execution. When there is another queued event for handling, as determined in step 2732, the next event is dequeued, in step 2734, and control returns to step 2706 to handle the next event. Otherwise, control returns to step 2704, where the client-site application waits for the occurrence of an event. In the currently described implementation of the currently disclosed system, the bulk of the computations are carried out by the server application, with the client-site application primarily orchestrating data downloads from external sources to the client-site application and data uploads to the server application, collecting data relevant to user requests for transmission to the server application, and displaying health-entropy-metric values and other information generated by the server application to the user. The interfaces provided by the client-site application to healthcare professionals, healthcare organizations, researchers, and research organizations may differ substantially from those provided to individual users.

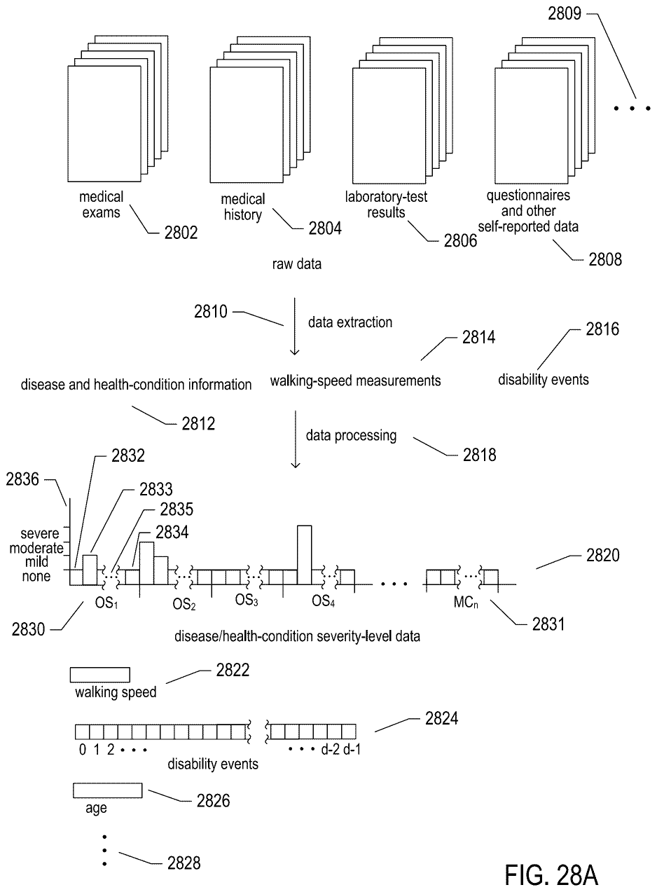

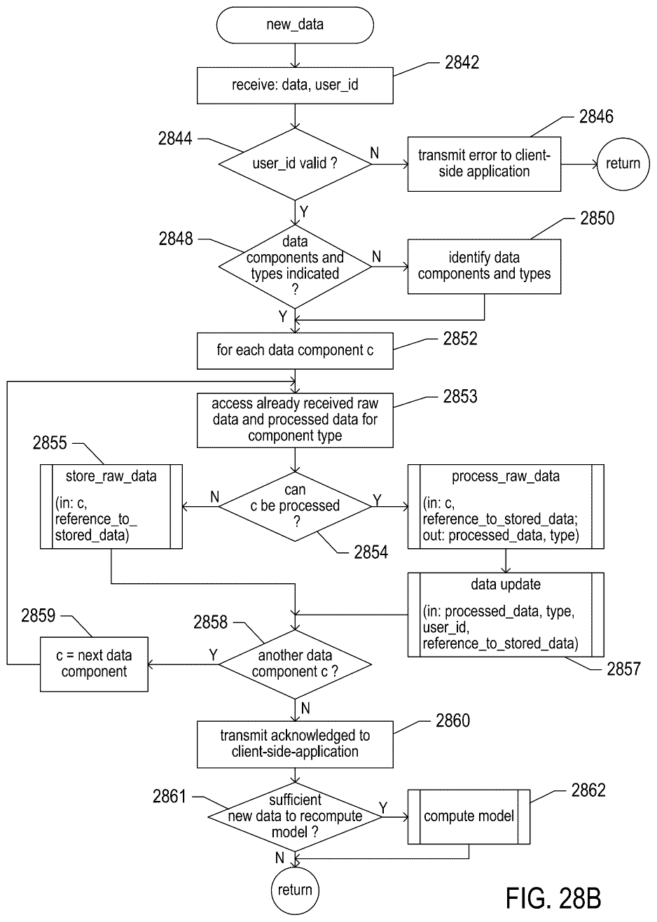

FIG. 28A illustrates the type of raw data and processed data that is used by the current system for generating computational models and using the computational models to generate health-entropy-metric values and other derived values. At the top of FIG. 28A, various types of raw data are illustrated. The raw data may include reports for medical exams 2802, medical-history and medications-history documents 2804, laboratory-test results 2806, and questionnaires and other self-reported data 2808, with ellipsis 2809 indicating that various other types of raw data may be used. The raw data may be obtained in electronic form, printed form, or accessed from databases and data archives. Various types of data-extraction methods are used 2810 to extract specific types of data from the raw data, including disease and health-condition information 2812, walking-speed measurements 2814, and the occurrences of various different disability events 2816 when new physical or cognitive disability measures are available. The extracted information is then processed 2818 to generate processed data that can be input to a routine model_adjustment, discussed below, to update the computational model and input to a routine compute_values, also discussed below, to compute health-entropy-metric values and other computed values for a particular individual based on data provided by the individual to the currently disclosed system.

In one implementation of the currently disclosed methods and systems, the processed data includes disease/health-condition severity-level data 2820, walking-speed data 2822, disability-events data 2824, and data about an individual, including individuals chronological age 2826 and additional information represented by ellipsis 2828. The disease/health-condition severity-level data 2820 includes data for each organ system (“OS”), such as OS₁ 2830, and each multi-organ-system condition (“MC”), such as MCₙ 2831. The data for each OS and MC comprises a severity level for each health condition or disease associated with the OS or MC. Thus, for example, for OS₁, each rectangular column 2832-2834, with ellipsis 2835 indicating that there may be additional health conditions and diseases associated with OS₁, represents a health condition or disease and the height of the rectangular column represents the severity level. As indicated along the vertical axis 2836, the severity levels include: none, mild, moderate, and severe. As discussed below, the disease/health-condition severity-level data 2820 provide a basis for determining the values of numerous clocks corresponding to OSs, MCs, the whole body, and a number of functionalities. The walking speed 2822 is a velocity that may be measured in a clinical setting or self-determined by a user. A user's normal walking speed has been found to be related to the user's age and the user's health entropy. The disability-events data 2824 can be thought of as a vector of bit flags indicating whether or not any of a number of disability events has occurred for a user in this data is generally obtained from a questionnaire or medical-exam report. The number of disability events is used to generate a disability index that, in turn, is used to determine a disability clock and disability biological age, as discussed below. It is important to note that the processed data illustrated in FIG. 28A represents the processed data generated for one implementation of the currently disclosed methods and systems. Alternative implementations may supplement the processed data with additional data types, use different processed data, or use certain of the illustrated types of processed data along with other types of processed data. Regardless of the exact types of processed data generated and used by an implementation of the currently disclosed methods and systems, the processed data is generated from data extracted from a variety of different data sources, as discussed above. The data represents not only information about diseases experienced by a user, but also sub-clinical health conditions and various other types of information. In other words, the currently disclosed methods and systems can access many different types of information and generate processed data of many types to provide a comprehensive basis for ascertaining a user's health entropy, which is something that is not provided or considered in generating currently available health metrics.

FIG. 28B provides a control-flow diagram for the new-data handler called in step 2616 of FIG. 26. In step 2842, the new-data handler receives data transmitted by a user along with an identifier for the user. The identifier may include a numeric or alphanumeric user_ID, in certain implementations, while in other implementations, the identifier may include additional credentials, biometric data, passwords, and other types of identifying information. In step 2844, the new-data handler determines whether the supplied identifying information is valid. If not, the new-data handler transmits an error to the client-site application in step 2846 before returning. Otherwise, in step 2848, the new-data handler determines whether the data includes information that allows the new-data handler to identify data components and datatypes within the received data. For example, this might include metadata, labels, and other such information that explains what has been transmitted. If not, then the new-data handler may use various methods, in step 2850, to identify the data components and datatypes included in the received data. In the for-loop of steps 2852-2859, each data component c in the received data is considered. In step 2853, the new-data handler accesses already received raw data and processed data for the component type and user. If any already received raw data and processed data for the component type along with the data component c extracted from the received data provides sufficient basis for processing the accumulated data to generate additional processed data, such as health-condition or disease severity-level data for a particular organ system or condition that spans multiple organ systems, the accumulated data is processed in step 2856 and used to update the stored processed data for the user in step 2857. Otherwise, in step 2855, the currently considered data component is stored as raw data for the user to use for subsequent data processing when sufficient data has been accumulated. When there is another data component that can be extracted from the received data, as determined in step 2858, the next data component is extracted, in step 2859, and control returns to step 2853 for processing the next data component. Otherwise, in step 2860, the new-data handler transmits an acknowledgment to the client-site application. In step 2861, the new-data handler determines whether there is now sufficient new data stored by the server application from multiple users to recompute the computational models used to generate health-entropy-metric values and other derived information. If so, then, in step 2862, the routine “compute model” is called. Thus, users may supply health information and other information to the server application in a piecemeal fashion, over time. The server application accumulates the received data and, when possible, processes the data to generate processed data that is stored for the user for use in generating health-entropy-metric values and other values derived from the stored, processed data. The server application stores the processed data obtained from users since the last computational-model update in order to periodically update or generate the computational models for the various health-entropy metrics and other computed values.

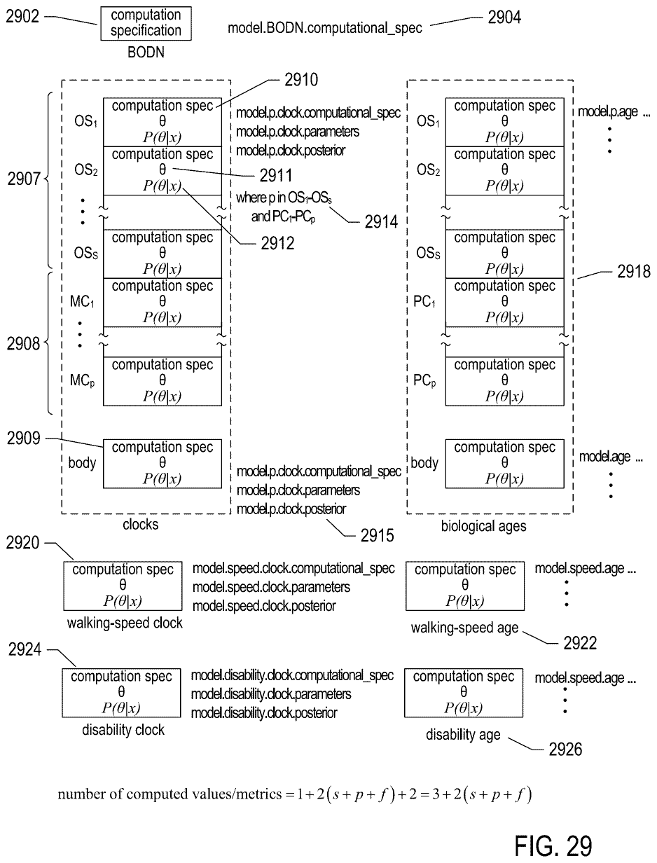

FIG. 29 illustrates the contents of the overall computational model used to generate health-entropy-metric values and other intermediate values from health data and other information furnished by users in one implementation of the currently disclosed system. The overall computational model includes numerous discrete computational models for generating each different health-entropy-metric value and other values used to compute the health-entry-metric value. A first computed value is the Body Organ Disease Number (“BODN”) 2902. A specification for computing the BODN comprises the computational model for the BODN. This computational model is not generally updated or refined as new data is received by the server application. In the following control-flow diagrams, the computational specification for the BODN is referred to by the expression 2904. There are numerous computed values referred to as “clocks” 2906. These clocks include clocks for various different organ systems 2907, clocks for multi-organ-system conditions 2908, and a whole-body clock 2909. The computational models for the clocks generally include a computation specification, such as computational specification 2910, generally an expression including parameter coefficients in terms that are added together to produce a metric value, a vector of current parameter values, such as parameter vector 2911, and the most recently computed posterior probability distribution that can be subsequently used as the prior for Bayesian-inference-based regression, such as posterior 2912. Expressions used in subsequent control-flow diagrams for these components of the computational model are provided as expressions 2914 and 2915. There are numerous health-entropy-metric values, or biological ages, for the various different organ systems, the multi-organ-system conditions, and the whole-body 2918. The computational models for each of these computed values generally include a computational specification, a current-parameter-value vector, and the last generated posterior probability distribution. In addition, there are clocks and health-entropy-metric values, or biological ages, computed for each of multiple different functionalities. In one implementation these include a walking-speed clock 2920, a walking-speed biological age 2922, a disability clock 2924, and a disability biological age 2926. Walking speed can be determined by a healthcare provider for a given individual or, in certain cases, by the individual himself or herself. Walking speed is known to be correlated with age and health entropy. The disability clock and disability biological age are based on a disability index that is determined via questionnaires and other types of data-collection methods. In one implementation, there are 47 different disability events or indications that an individual may have experienced or exhibited, and indications of whether or not each of these disability events have been experienced or exhibited are used to generate a disability-index value that is then used for computing the disability clock and disability age. In the described implementation, there are s 11 different organ systems, p=2 different multi-organ-system conditions, and ƒ=2 different functionalities, and thus 3+2 (s+p+f)=33 values computed by the overall computational model: the BODN, 11 clocks for organ systems, two clocks for conditions that span multiple organ systems or comprise multiple different types of events, a whole-body clock, and two clocks for functionalities, 11 biological ages for organ systems, two biological ages for multi-organ-system conditions, a whole-body biological age, and two biological ages for functionalities. In the described implementation, the functionalities include walking speed and the disability index, the multi-organ-system conditions include cancer and cerebrovascular accidents, and the organ systems include the cardiovascular system, the renal system, the metabolic system, the gastrointestinal and liver system, the respiratory system, the thyroid system, the hematopoietic system, oral health, the musculoskeletal system, the sensory system, and the central nervous system.

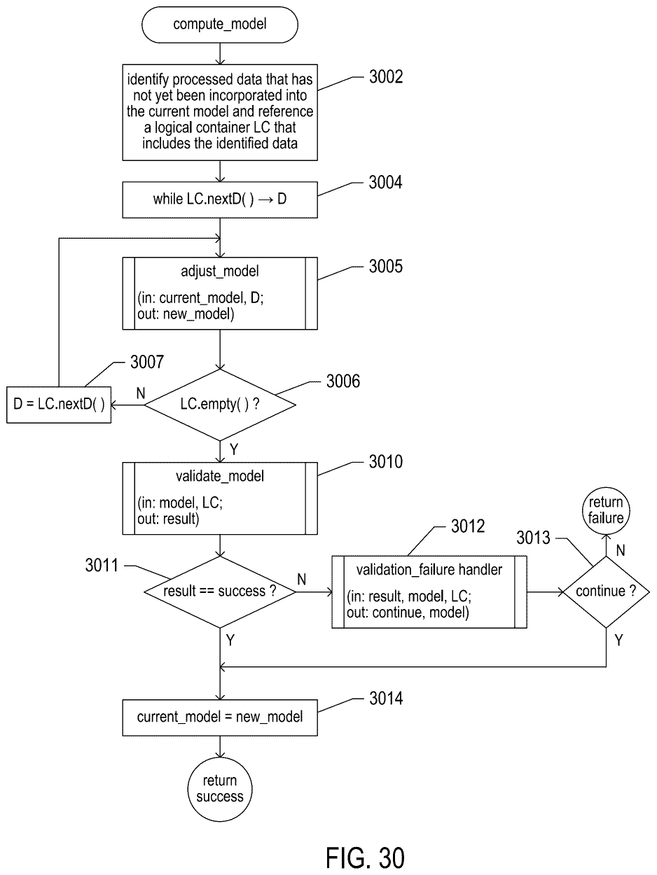

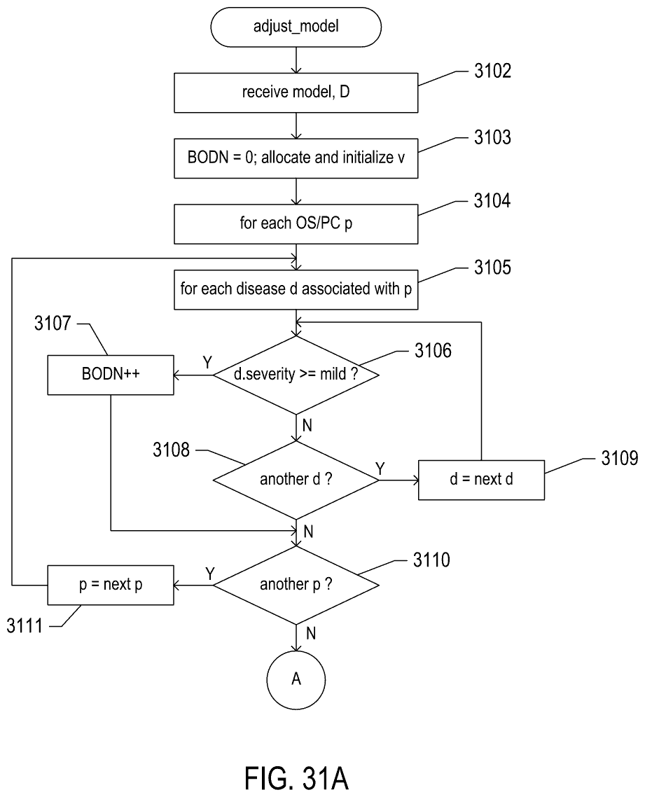

FIG. 30 provides a control-flow diagram for the routine compute model called in step 7822 of FIG. 28. This routine updates the computational models for the various computed values generated by the currently disclosed system, including the health-entropy-metric values, once sufficient new data has been collected by the currently disclosed system. In step 3002, the routine compute_model identifies stored processed data that has not yet been incorporated into the current model and references the identified processed data as a logical container LC. In the while-loop of steps 3004-3007, the routine compute_model extracts a next data set or data vector D from LC and inputs the data set or data vector D, along with a reference to the current model, to a routine “adjust_model,” which uses D to generate and return a new model including new parameters and posterior distributions. When the LC is not empty, as determined in step 3006, a next data vector is extracted from the LC in step 3007 and control returns to step 3005. Otherwise, in step 3010, a routine “validate_model” is called to validate the new model. There are different methods for model validation, including using data left out of the model-generation process to generate new parameter values and checking whether those new computed values are consistent with credible intervals computed from the posterior distributions. Alternatively, predicted health-entropy-metric values can be checked against ground-truth values obtained by other methods or from other sources. When validation is successful, as determined in step 3011, the routine compute_model updates the current model to the new model, in step 3014, and returns with a success indication. Otherwise, a validation-failure handler is called in step 3012. The validation-failure handler may attempt to generate the new model using one or more different prior distributions or likelihood distributions, may analyze the data for outlier data or erroneous data that may have contributed to a defective new model, and/or take other types of actions in attempting to try again to generate an acceptable new model. If the validation-failure handler returns an indication to continue, as determined in step 3013, control flows to step 3014. Otherwise, the routine compute_model returns with a failure indication. Model-generation failures are handled at higher levels.

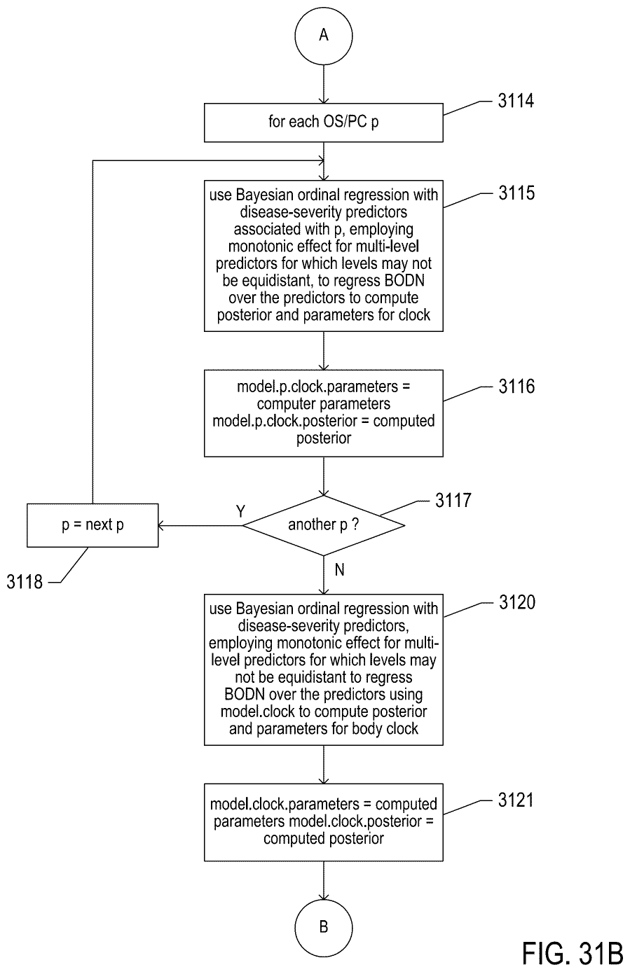

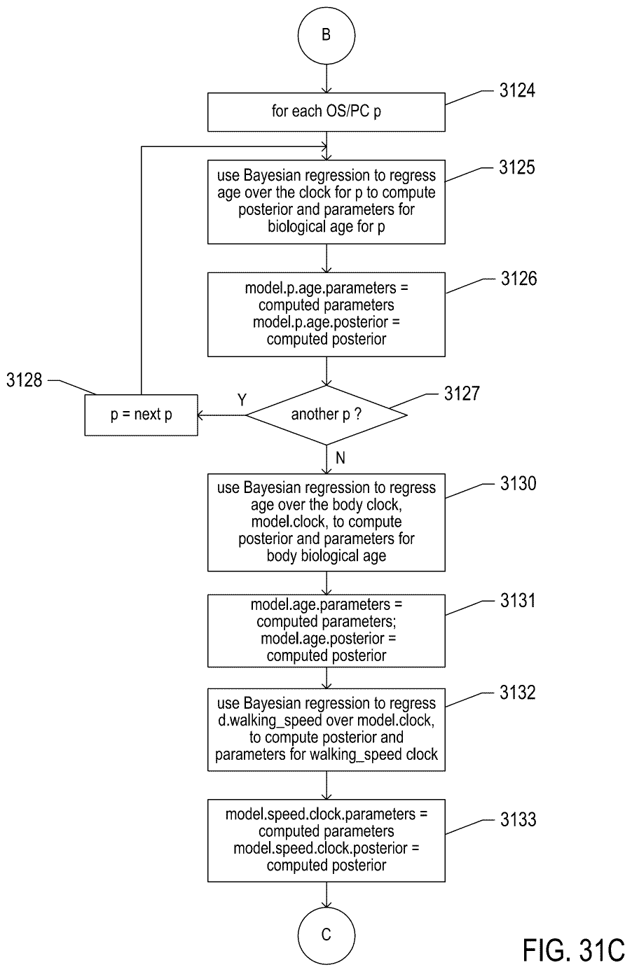

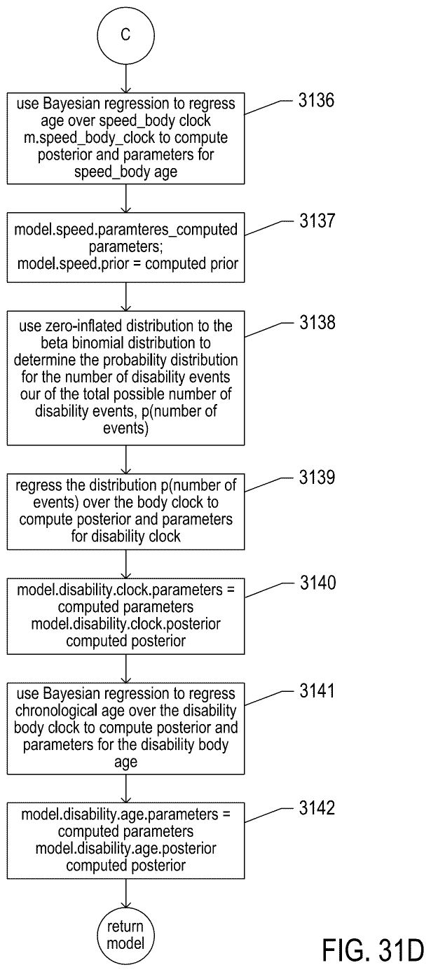

FIGS. 31A-D provide a control-flow diagram for the routine adjust_model called in step 3005 of FIG. 30. This routine updates the model referenced by the received argument model using the received data set or data vector D. In step 3102, the routine adjust_model receives a reference to a model and a data vector D. In step 3103, the routine adjust_model sets local variable BODN to 0. The outer for-loop of steps 3104-3111 In the outer for-loop of steps 3104-3111, the routine adjust_model considers each organ system (“OS”) and condition that spans multiple organ systems (“MC”) p. Note p is a local variable that references an OS or MC. In the inner for-loop of steps 3105-3109, the routine adjust_model considers each disease or condition d associated with p. When the severity level associated with the currently considered disease or condition d is greater than or equal to mild, as determined in step 3106, local variable BODN is incremented, in step 3107, and the inner for-loop terminates. Otherwise, when there is another disease or condition associated with the currently considered OS or MC p, as determined in step 3108, dis set to the next disease or condition, in step 3109, and control returns to step 3106. Otherwise, in step 3110, the routine adjust_model determines whether there is another OS or MC. When there is another OS or MC to consider, p is set to the next OS or MC to consider, in step 3111, and control returns to step 3105. The BODN is thus computed as the number of OSs and MCs associated with at least one disease or condition with a severity level greater than or equal to mild.

Turning to FIG. 31B, in the for-loop of steps 3114-3118, each OS and MC p is again considered. In step 3115, Bayesian ordinal regression is used with the disease-severity predictors associated with p and employing the monotonic effect for multi-level predictors for which levels may not be equidistant to regress BODN over the predictors to compute the posterior and then the parameters for the clock corresponding to p. The predictors can also be continuous or binary. Then, in step 3116, the model is updated to include the computed parameters and posterior. Thus, the for-loop of steps 3114-3118 uses Bayesian ordinal regression to compute the clock parameters, and posteriors for the clocks associated with OSs and MCs (2907 and 2908 in FIG. 29). In steps 3120 and 3121, the routine adjust_model similarly computes the posterior and then the parameters, using Bayesian ordinal regression, for the body clock and updates the model to include the parameters and posterior. Thus, steps 3120-3121 update the model for the body clock (2909 in FIG. 29).

Turning to FIG. 31C, in the for-loop of steps 3124-3128, the routine adjust_model uses Bayesian regression to regress chronological age over the clock for each OS and MC p to compute the posterior and parameters for the biological age metric for p and then update the model with the computed parameters and posterior. Thus, the for-loop of steps 3124-3128 computes the posterior and parameters for the biological age metric for each of the OSs and MCs (contained in 2918 in FIG. 29). In steps 3130-3131, the routine adjust_model computes the posterior and parameters for the body biological age (contained in 2918 in FIG. 29) and stores the computed parameters and posterior in the model. In step 3132-3133, the routine adjust_model computes the posterior and parameters for the walking-speed clock using Bayesian regression over the body clock and stores the computed parameters and posterior in the model. Note that chronological age is used to estimate the biological age in these regressions.

Turning to FIG. 31D, in steps 3136-3137, the routine adjust_model computes the posterior and parameters for the walking-speed biological age (2922 in FIG. 29) using Bayesian regression to regress chronological age over the walking-speed clock and stores the computed parameters and posterior in the model. In step 3138, the routine adjust_model uses a zero-inflated distribution to the beta binomial distribution to determine a probability distribution p(number of events) for the number of disability events out of the total possible number of disability events. In step 3139, the routine adjust_model regresses the distribution p(number of events) over the body clock to compute the posterior and parameters for the disability clock and stores the parameters and posterior in the model in step 3140. Finally, in step 3141-3142, the routine adjust_model uses Bayesian regression to regress chronological age over the disability clock to compute the posterior and parameters for the disability age and stores the computed parameters and posterior in the model.

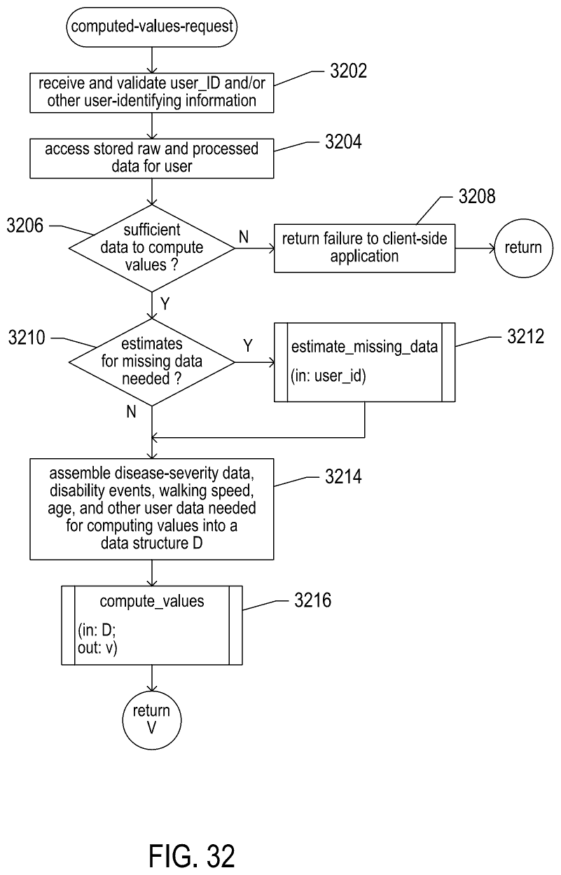

FIG. 32 provides a control-flow diagram for the routine computed_values_request called in step 2620 of FIG. 26. In step 3202, the routine computed values_request_received a user_ID and/or other identifying information for a user, as in steps 2842, 2844, and 2846 in FIG. 28B. Only when the user-identifying information is determined to be valid does the routine computed_values_request continue to step 3204, where the routine accesses stored raw and processed data for the user. When there is insufficient processed data for the server application to compute health-entropy-metric values for the user, as determined in step 3206, the server application returns a failure indication to the user's client-side application, in step 3208, and returns. Otherwise, the routine computed_values_request determines, in step 3210, whether it is necessary to insert estimates for missing data in order to facilitate health-entropy-metric-value calculations and, if so, calls the routine estimate_missing_data, in step 3212, to supplement the processed data available for the user. In certain cases, the server application may determine that only certain of the health-entropy-metric values can be computed and subsequently computes only those values for which sufficient processed data is available. There are a variety of techniques and methods for determining when it is possible to compute metric values even though all of the desired processed data is not available for the computations and for computing those values despite processed-data deficiencies. In step 3214, the routine computed_values_request assembles the most current processed data for the user into a data structure D and supplies the data structure as an argument to the routine compute_values, in step 3216. The routine compute_values returns a data structure v containing computed health-entropy-metric values and other computed values that is then returned by the server application to the client-side server for display to the user, for transmission by the user to downstream entities, and for use by the user in a variety of tasks and activities, as discussed above.

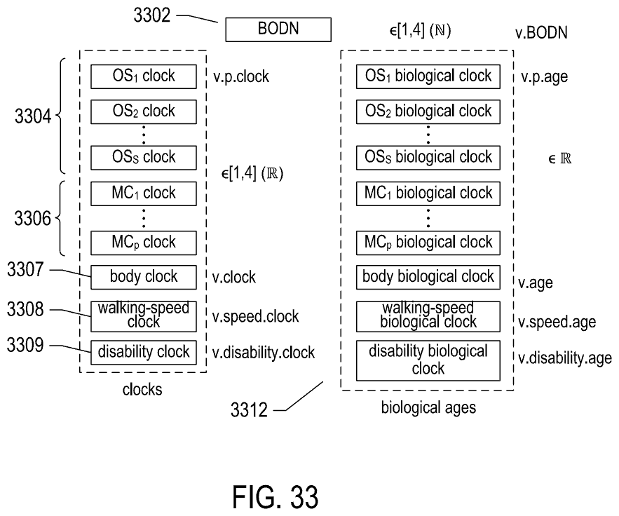

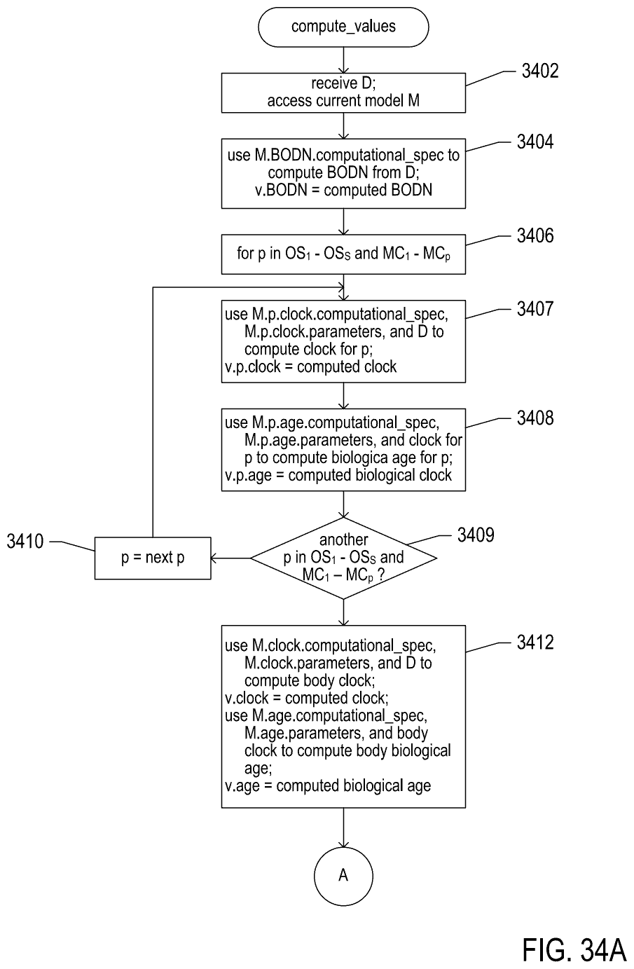

FIG. 33 illustrates the health-entropy-metric values and other computed values that are calculated by the server application, using the above-described computational models, incorporated into the above-mentioned data structure v, and returned to a user. The body organ disease number (“BODN”) 3302 is an integer value in the range [1, 14] and stored in v.BODN within data structure v. There are clocks for each organ system 3304 and multi-organ-system condition 3306 as well as a clock for the whole body 3307, a walking-speed clock 3308, and a disability clock 3309. Using p to refer to both organ systems and multi-organ-system conditions, the data structure v stores the organ-system and multi-organ-system clocks in v.p.clock in data structure v. The whole-body clock is stored in v.clock, the walking-speed clock is stored in v.speed.clock, and the disability clock is stored in v.disability.clock. Each clock has a real value in the range [1, 14]. This is because, as discussed above, the clocks are obtained by Bayesian ordinal regression of the BODN over processed-data values, such as the health-condition and disease severity levels associated with organ systems and multi-organ-system conditions. For each clock, there is a corresponding biological age 3312. Using p to again refer to both organ systems and multi-organ-system conditions, the data structure v stores the organ-system and multi-organ-system biological ages in v.p.age in data structure v. The whole-body biological age is stored in v.age, the walking-speed biological age is stored in v.speed.age and the disability biological age is stored in v.disability.age. The biological ages are real numbers that extend up through biological ages significantly greater than chronological ages encountered in users to reflect increases in health entropy above normal or average values.

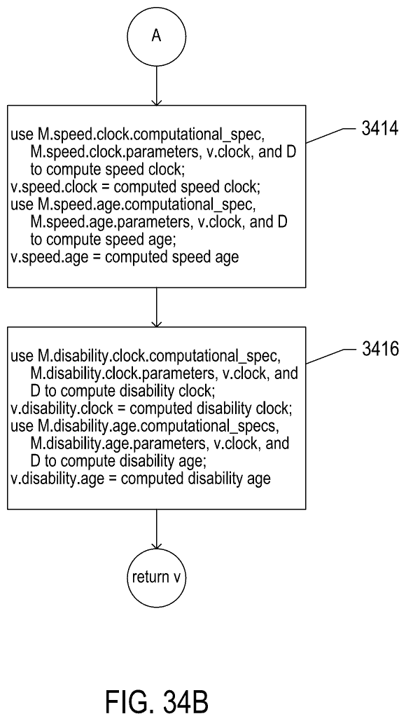

FIGS. 34A-B provide a control-flow diagram for the routine compute_values called in step 3216 of FIG. 32. This routine computes the health-entropy-metric values and other values included in data structure v and discussed above with reference to FIG. 33. In step 3402, the routine compute_values receives the data structure D containing processed data from which health-entropy-metric values and other values are computed as well as a reference M to the current model maintained by the server application. As discussed above, the server application may choose not to compute certain of the values when there is insufficient processed data to do so, but, in the interest of simplicity and brevity, the control-flow diagrams for the routine compute_values show computation of all of the values. In step 3404, the routine compute_values uses the computational specification for the BODN calculation in the current model, M.computational_spec to compute the value of BODN from the health-condition and disease data stored in D and stores the computed value in v.BODN. The BODN is the number of organ systems and multi-organ-system conditions associated with at least one health condition or disease with severity levels greater than or equal to mild.

In the for-loop of steps 3406-3410, the routine compute_values considers each organ system and multi-organ-system condition p. In step 3407, the computational specification for the clock for the currently considered organ system or multi-organ-system condition, the current parameter values for the computational specification, and the relevant processed data in data structure D are used to compute the value for the clock for organ system or multi-organ-system condition p. In step 3408, the computational specification for the biological age for the currently considered organ system or multi-organ-system condition, the current parameter values for the computational specification, and the relevant processed data in data structure D are used to compute the biological-age value for organ system or multi-organ-system condition p. When there is another organ system or multi-organ-system condition to consider, as determined in step 3409, control flows to step 3410, where p is set to reference the next organ systems or multi-organ-system condition, with control then flowing back to step 3407. In step 3412, the routine compute_values computes the whole-body clock and whole-body biological age using the computational specifications and parameters stored in the current model. Turning to FIG. 34B, in step 3414, the routine compute_values computes the walking-speed clock and walking-speed biological age using the computational specifications and parameters stored in the current model. Finally, in step 3416, the routine compute_values computes the disability clock and disability age using the computational specifications and parameters stored in the current model.

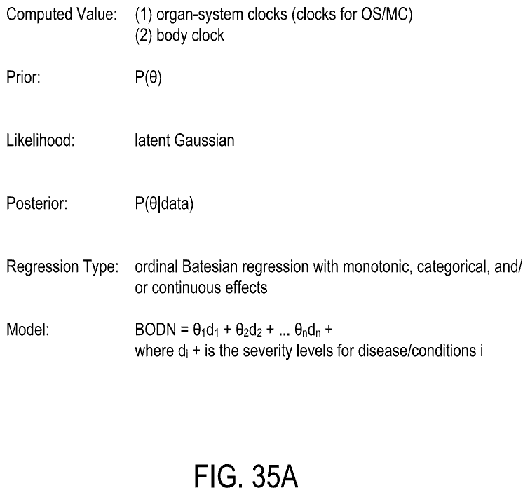

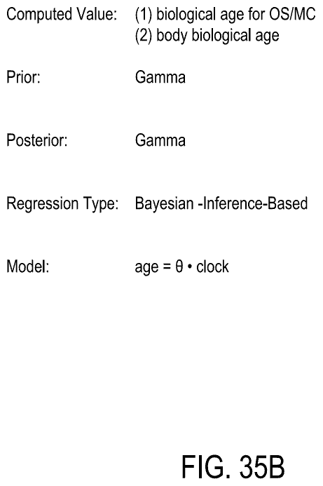

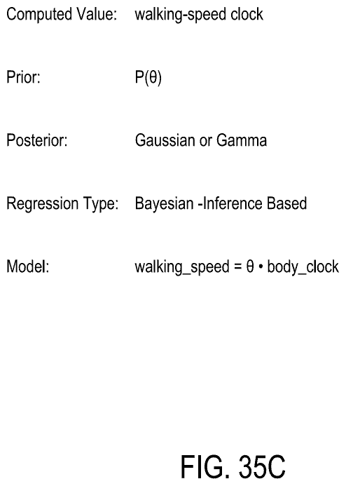

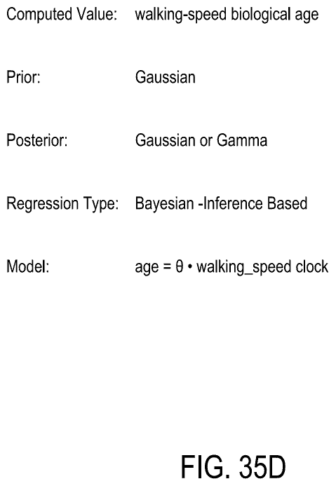

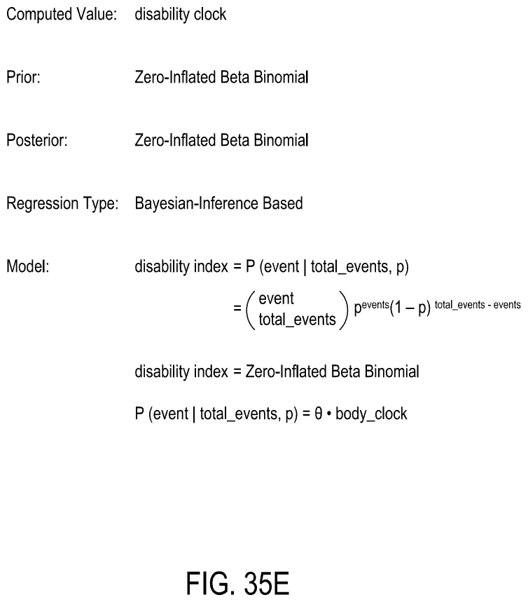

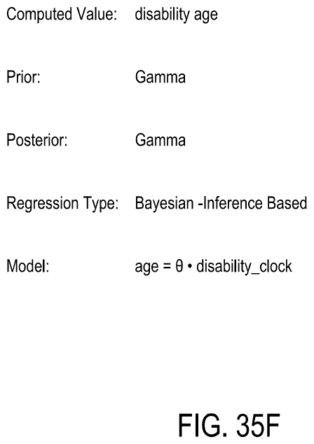

FIGS. 35A-F provide details for each of the Bayesian-inference-based regressions used to generate the computational models for the various health-entropy-metric values and other computed values by the currently disclosed methods and systems. At the top of each figure, the value computed by the model generated by the regression is indicated. The types of the prior and posterior distributions for the Bayesian-Inference-Based regressions are shown, followed by an indication of the Bayesian-Inference-Based Regression type. Finally, a model is shown for the regression. For the organ-system clocks and the body clock, as shown in FIG. 35A, the integer-valued BODN is regressed over the sum of multiple terms, each term the product of a disease-severity level or health-condition severity level and a parameter. Once the parameters are determined, they can then be used to compute a real-valued BODN from the disease-severity level and health-condition severity levels of the diseases and health conditions associated with the organ system, in the case of an organ-system clock, or the disease-severity level and health-condition severity levels of the diseases and health conditions associated with all of the considered diseases and health conditions, for the whole-body clock. FIG. 35B shows the regression for the organ-system and whole-body clocks. Chronological age is regressed over the product of a parameter and the organ-system and whole-body clock. Once a value for the parameter is determined by regression, it can be used to calculate the biological age as the product of the parameter and the clock. Similar comments apply to FIGS. 35C, 35D, and 35F. In FIG. 35E, the disability clock involves first determining the disability index using a Zero-Inflated Beta Binomial distribution and then regressing the probability computed from that over the product of a parameter and the body clock.

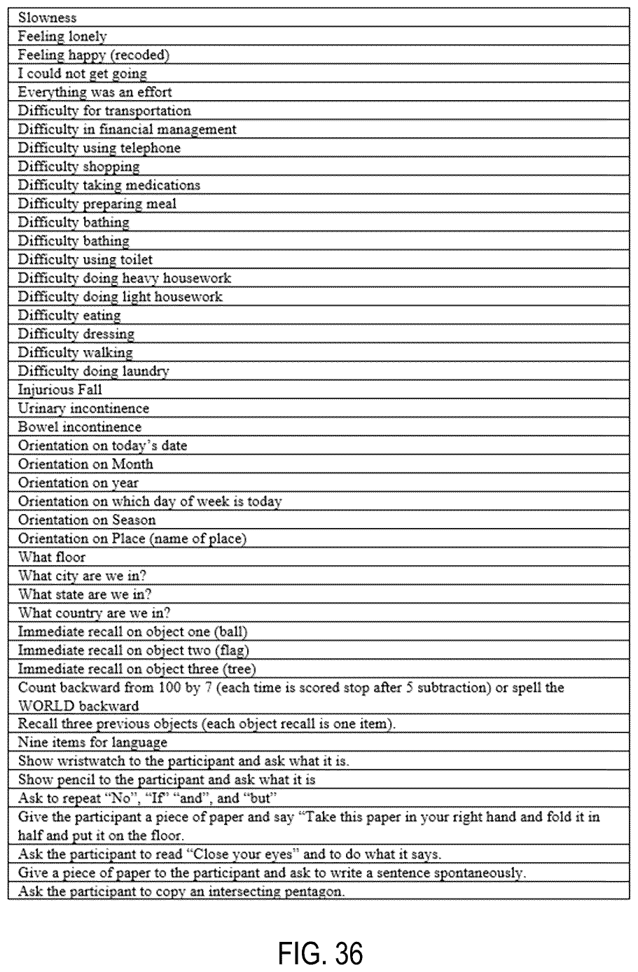

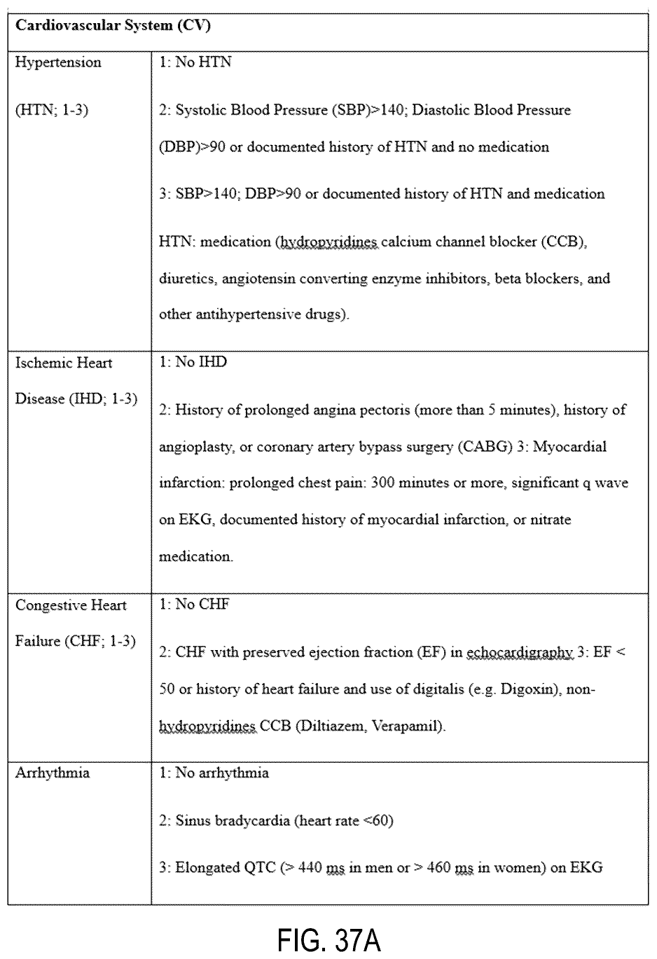

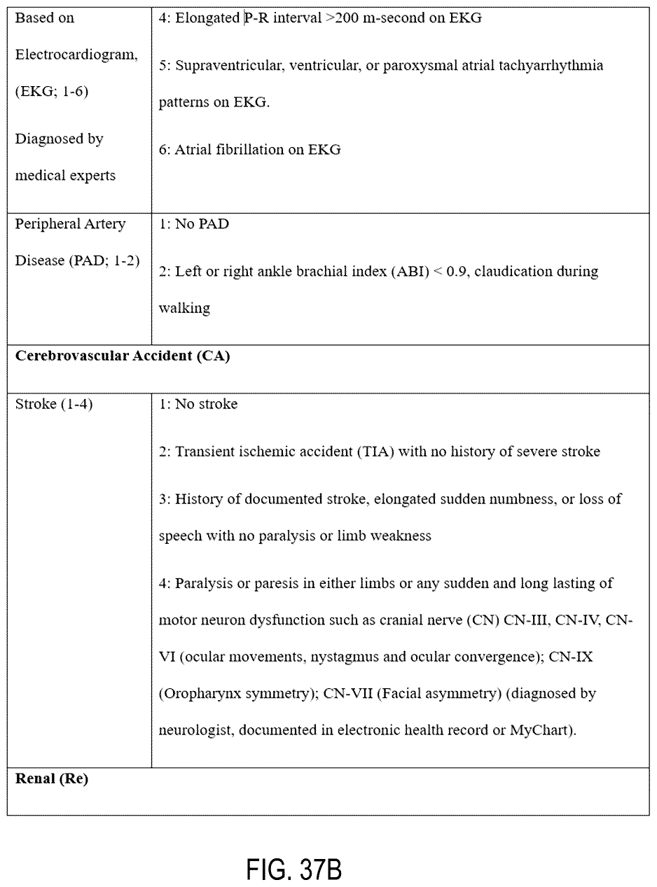

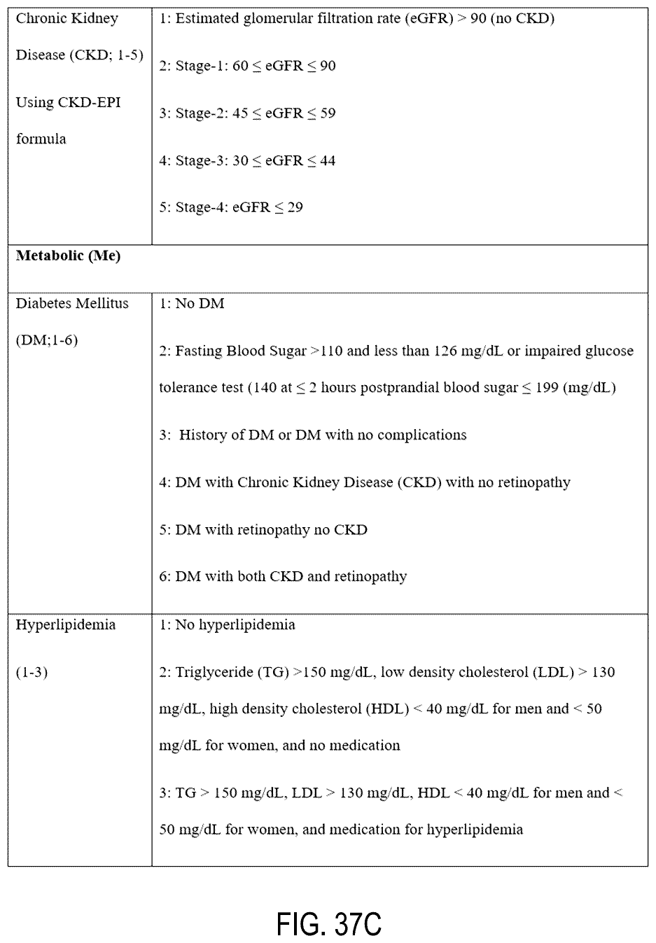

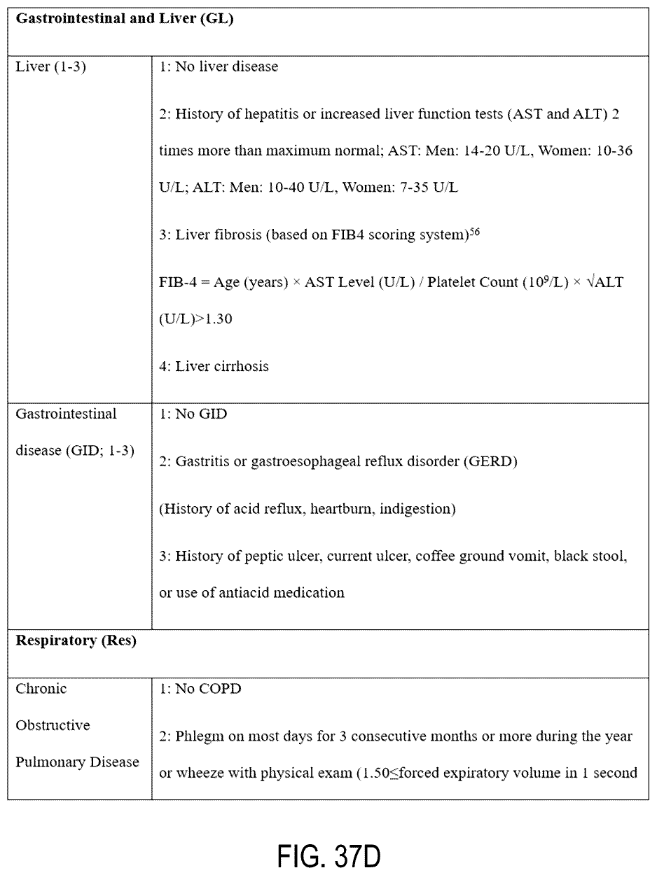

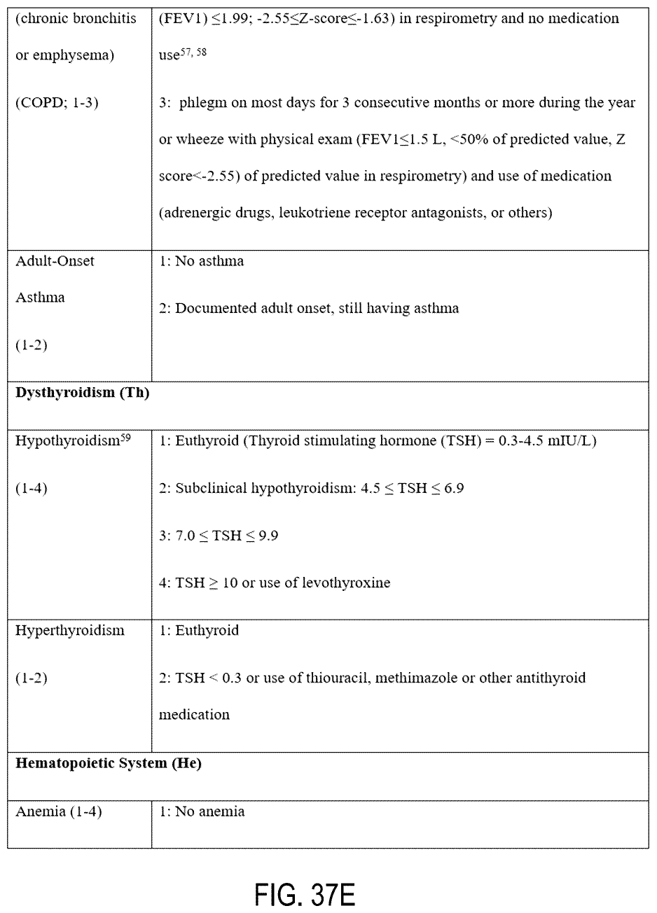

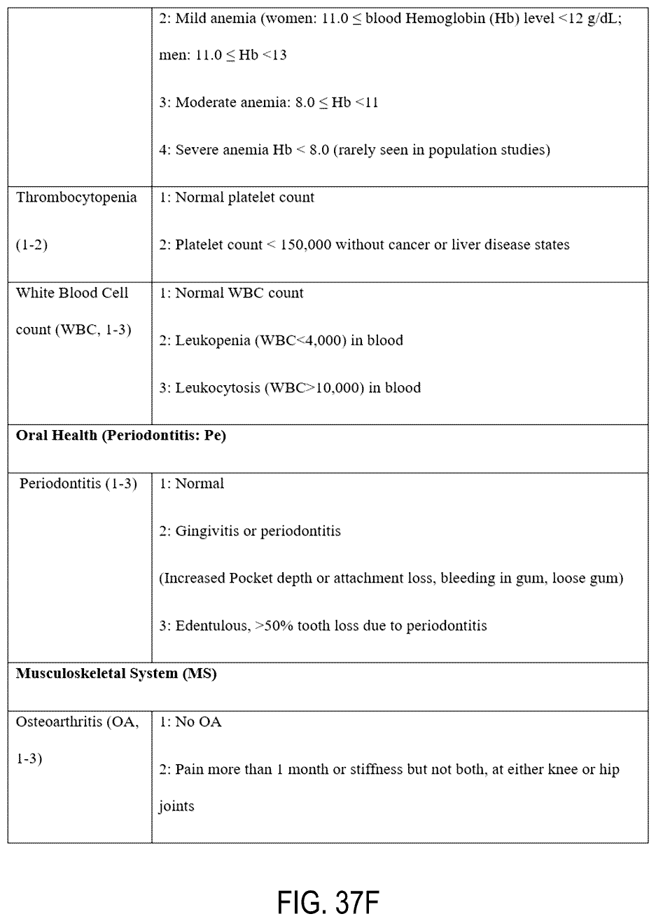

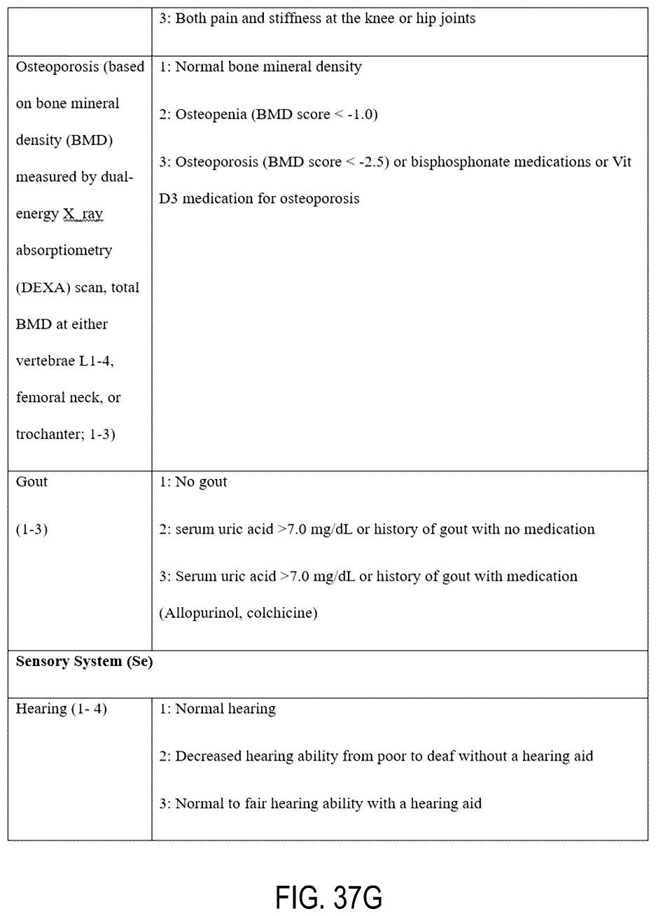

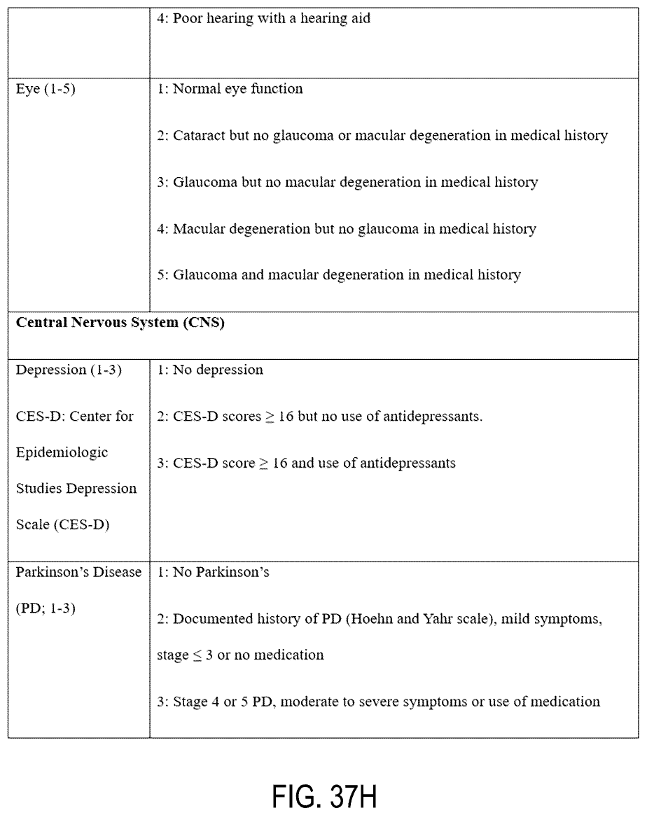

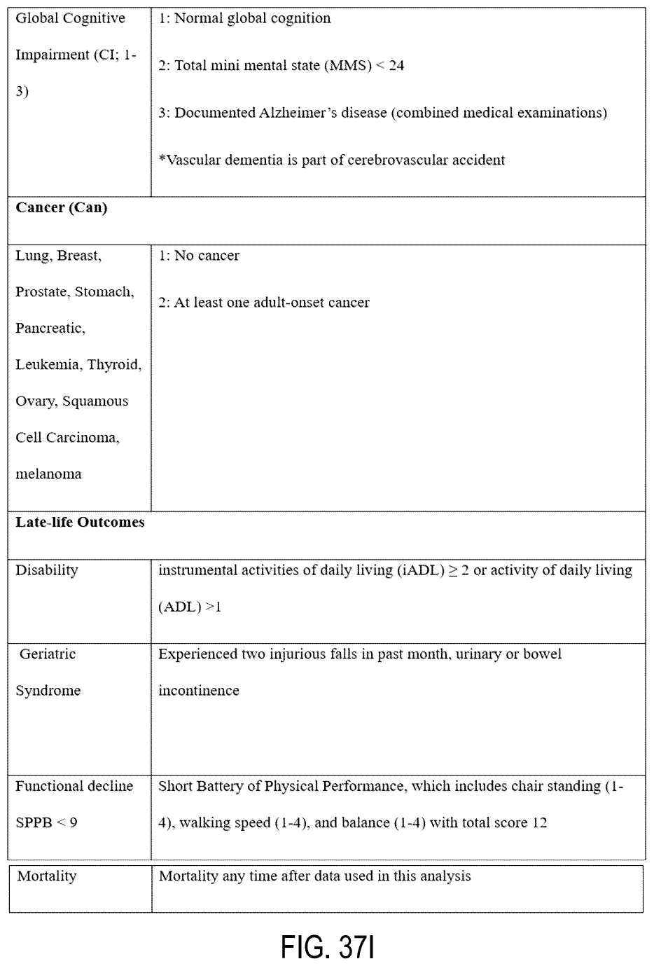

FIG. 36 shows the disability events used in one implementation of the currently disclosed methods and systems to generate the disability index, disability clock, and disability biological age. FIGS. 37A-I show the organ systems and multi-organ-system conditions, and associated health conditions and diseases, used in one implementation of the currently disclosed methods and systems.

The present invention has been described in terms of particular embodiments, but it is not intended that the invention be limited to these embodiments. Modifications within the spirit of the invention will be apparent to those skilled in the art. For example, any of many different implementations of the currently disclosed methods and systems can be obtained by varying various design and implementation parameters, including modular organization, control structures, data structures, hardware, operating system, and virtualization layers, automated orchestration systems, virtualization-aggregation systems, and other such design and implementation parameters. As noted above, in the disclosed implementation, the bulk of computations are performed by the data-center or cloud-computing-facility resident server application, but the distribution of computation may differ in alternative implementations. As one example, the server application could periodically push the current computational model to user computers so that the health-entropy-metric values and other computed values could be computed locally on user computers. In alternative implementations, different sets of organ systems and multi-organ-system conditions may be used for data collection and metric-value generation. In addition, in alternative implementations, for functionalities in addition to, or other than, walking speed and disability may be used for data collection and metric-value generation. A variety of different computational-model-generation methodologies are possible in addition to those used in the currently described implementation. The currently disclosed methods and systems are intended to provide flexibility and types and quantities of data that can be processed and incorporated into the calculation of the various different health-entropy-metric values and other metric values. The discussion has focused on receiving, by the server application, data from individual users and returning health-entropy-metric values and other computed values to the individual users. However, in many cases, when information is transmitted to researchers and research organizations, it is not individual health-entropy-metric values that are most useful, but instead information derived from the computational models used to generate them.

1. An automated system that collects and processes data, generates health-entropy-metric-value computational models using the processed data, and uses the computational models to generate health-entropy-metric values and derived information that the automated system transmits to individuals and organizations for health-care provision, research, and personal-health evaluation and monitoring, the automated system comprising:
a server application, implemented in multiple processing entities within one or more data centers and/or cloud-computing facilities, that
receives multiple types of information from multiple information sources, including multiple individual users,
extracts health-related, physiology-related, and personal data from the received multiple types of information, including disease data and sub-clinical-health-condition data,
processes the data to generate input data,
generates and periodically adjusts multiple computational models for multiple health-entry metrics, including body biological age and multiple organ-system-specific biological ages,
transmits health metrics, generated by inputting input data obtained by processing data received from individual users to the multiple computational models, to users who use the health metrics to evaluate and monitor personal-health, and
transmits health metrics and information derived from the multiple computational models and received information to medical professionals and medical organizations to use in diagnosis and treatment and to researchers to use in one or more of drug development, biomarker selection, medical research, and public-health monitoring; and
a client-side application that runs on user computers to collect information from one or more users, transmit information and requests to the server application and to receive, from the server application, one or more of health metrics, additional computed values, and information derived by the server application from the multiple computational models and information received by the server application.

2. The automated system of claim 1 wherein the multiple types of information received from multiple information sources by the server application includes:
medical-examination reports;
laboratory-test results;
medical histories;
medication histories;
user responses to prompts by the client-side application;
questionnaires;
user-self-reported health information.

3. The automated system of claim 1 wherein the input data for an individual, generated by the server application by extracting health-related, physiology-related, and personal data from the received multiple types of information, comprises:
disease and health-condition severity levels for diseases and health conditions related to each of multiple organ systems and multi-organ-system conditions, wherein severity levels include none, mild, moderate, and severe;
indications of which of a number of disability-related events have been experienced by the individual;
the individual's normal walking speed; and
personal information for the individual, including age.

4. The automated system of claim 1 wherein the organ systems for which metrics are generated by the server application include:
the cardiovascular system;
the renal system;
the metabolic system;
the gastrointestinal and liver system;
the respiratory system;
the thyroid system;
the hematopoietic system;
the oral health system;
the musculoskeletal system;
the sensory system; and
the central nervous system.

5. The automated system of claim 1 wherein the multi-organ-system conditions for which metrics are generated by the server application include:
cancer; and
cerebrovascular accidents.

6. The automated system of claim 1 wherein the functionalities for which metrics are generated by the server application include:
walking speed; and
disability.

7. The automated system of claim 1 wherein the health metrics and other computed values generated by inputting input data obtained by processing data received from individual users to the multiple computational models include:
a body-organ disease number;
a clock for the whole body and for each organ system, multi-organ-system condition, and functionality; and
a biological age for the whole body and for each organ system, multi-organ-system condition, and functionality.

8. The automated system of claim 7 wherein the body-organ disease number is computed as the number of organ-system and muti-organ-system conditions associated with one or more diseases or health conditions with a severity level greater than or equal to mild.

9. The automated system of claim 8 wherein the computational model for the clock for each organ system and multi-organ-system condition comprises the sum of multiple terms, each term comprising a severity level multiplied by a parameter, wherein the severity level is associated with a disease or health condition associated with the organ system or multi-organ-system condition.

10. The automated system of claim 9 wherein the parameter values are obtained by Bayesian-inference-based regression over the body-organ disease number using input data generated from information provided to the server application by multiple individuals.

11. The automated system of claim 8 wherein the computational model for the biological age for each organ system and multi-organ-system condition comprises the clock value computed for the organ system or multi-organ-system condition multiplied by a parameter.

12. The automated system of claim 11 wherein the parameter value is obtained by Bayesian-inference-based regression over chronological age using input data generated from information provided to the server application by multiple individuals.

13. The automated system of claim 8
wherein the computational model for the whole-body clock comprises the sum of multiple terms, each term comprising a severity level multiplied by a parameter, wherein the severity level is associated with a disease or health condition associated with an organ system or a multi-organ-system condition, and wherein the multiple terms include a term for each disease and health condition associated with each organ system and multi-organ-system condition; and
wherein the parameter value is obtained by Bayesian-inference-based regression over the body-organ disease number using input data generated from information provided to the server application by multiple individuals.

14. The automated system of claim 13
wherein the computational model for the whole-body biological age comprises the whole-body clock multiplied by a parameter; and
wherein the parameter value is obtained by Bayesian-inference-based regression over the BODN using input data generated from information provided to the server application by multiple individuals.

15. The automated system of claim 13
wherein the computational model for the walking-speed clock comprises a walking speed multiplied by a first parameter;
wherein the first parameter value is obtained by Bayesian-inference-based regression over the body clock using input data generated from information provided to the server application by multiple individuals;
wherein the computational model for the walking-speed biological age comprises the walking-speed clock multiplied by a second parameter; and
wherein the second parameter value is obtained by Bayesian-inference-based regression over chronological age using input data generated from information provided to the server application by multiple individuals.

16. The automated system of claim 13
wherein the computational model for the walking-speed clock comprises a walking speed multiplied by a first parameter;
wherein the first parameter value is obtained by Bayesian-inference-based regression over the body clock using input data generated from information provided to the server application by multiple individuals;
wherein the computational model for the walking-speed biological age comprises the walking-speed clock multiplied by a second parameter; and
wherein the second parameter value is obtained by Bayesian-inference-based regression over chronological age using input data generated from information provided to the server application by multiple individuals.

17. The automated system of claim 15
wherein a disability index is computed by a binomial distribution of a number of experienced disability events;
wherein the computational model for the disability clock comprises the disability index multiplied by a first parameter;
wherein the first parameter value is obtained by Bayesian-inference-based regression over the disability index using input data generated from information provided to the server application by multiple individuals;
wherein the computational model for the disability biological age comprises the disability clock multiplied by a second parameter; and
wherein the second parameter value is obtained by Bayesian-inference-based regression over chronological age using input data generated from information provided to the server application by multiple individuals.

18. A method that generates health-entropy-metric values for an individual, the method comprises:
receiving information relevant to the individual and the individual's health;
extracting health-related, physiology-related, and personal data from the received information, including disease data and sub-clinical-health-condition data,
processing the data to generate input data,
determining, from the input data, a body-organ disease number;
using computational models for multiple clock metrics to generate multiple clock-metric values;
using computational models for multiple biological-age metrics to generate biological-age-metric values; and
transmitting the body-organ disease number, multiple clock-metric values and multiple biological-age metrics to the individual to enable the individual to monitor medical treatments, monitor the individual's health, to evaluate and adjust diet, habits, and other lifestyle choices, and to efficiently access health care.

19. The method of claim 18
wherein the multiple clock-metric values include a clock-metric value for each of the individual's organ systems, including the individual's cardiovascular system; the individual's renal system; the individual's metabolic system; the individual's gastrointestinal and liver system; the individual's respiratory system; the individual's thyroid system; the individual's hematopoietic system; the individual's oral health system; the individual's musculoskeletal system; the sensory system; and the individual's central nervous system;
wherein the multiple clock-metric values include a clock-metric value for each of the individual's multi-organ-system conditions, including any cancer experienced by the individual and cerebrovascular accidents experienced by the individual; and
wherein the multiple clock-metric values include a clock-metric value for each of the individual's functionalities including the individual's walking speed and disability; and
wherein the multiple biological-age-metric values include a biological-age-metric value corresponding to each clock-metric value.

20. The method of claim 18
wherein the computational models each comprises the sum of multiple terms, each term comprising a severity level multiplied by a parameter, and
wherein the values of the parameters of the computational models are obtained by Bayesian-inference regression using input data generated from information provided to the server application by multiple individuals.
